(19) United States
(12) Patent Application Publication
(10) Pub. No.: US 2002/0040276 A1
(43) Pub. Date: Apr. 4, 2002

(54) Accessibility correction factors for electronic models of cytochrome P450 metabolism

(75) Inventors: Todd J.A. Ewing, Newark, CA (US); Jean-Pierre Kocher, Mountain View, CA (US); Hung Tieu, Alameda, CA (US); Kenneth R. Korzekwa, Mountain View, CA (US)

Correspondence Address: BEYER WEAVER & THOMAS LLP, P.O. BOX 778, BERKELEY, CA, 94704-0778, US

(73) Assignee: Camitro Corporation

(21) Appl. No.: 09/902,470

(22) Filed: Jul. 9, 2001

Related U.S. Application Data
(63) Continuation-in-part of application No. 09/613,875, filed on Jul. 10, 2000.
(60) Provisional application No. 60/217,227, filed on Jul. 10, 2000.

(51) Int. Cl. G06F 19/00; G01N 33/48; G01N 33/50

(52) U.S. Cl. 702/19

(57) ABSTRACT

Accessibility correction factors may be used to modify values predicted by models of electronic component substrate reactivity. Most of the correction factors described herein pertain to either steric or orientation effects on substrate accessibility. The correction factors may be derived from one or more “descriptors” of the substrate structure. Each group of descriptors and associated correction factor pertain to a particular site on the substrate. Examples of such descriptors include site polarity, protrusion, partial surface area, partial charge, etc. Often the correction factor is a function of multiple descriptors. The function may be an expression comprising multiple terms, each representing the weighted contribution of a particular descriptor. In other embodiments, the correction factor is simply a descriptor or a descriptor multiplied by a coefficient or other function.

### CROSS-REFERENCE TO RELATED APPLICATIONS

This patent application claims priority under 35 U.S.C. § 119(e) from U.S. Provisional Application No. 60/217,227 “Accessibility Correction Factors for Quantum Mechanical and Molecular Models of Cytochrome P450 Metabolism,” and this patent application is a continuation in part of U.S. patent application Ser. No. 09/613,875, “Relative Rates of Cytochrome P450 Metabolism,” both filed Jul. 10, 2000. This application is related to U.S. patent application Ser. No. 09/368,511, “Use of Computational and Experimental Data to Model Organic Compound Reactivity in Cytochrome P450 Mediated Reactions and to Optimize the Design of Pharmaceuticals,” filed Aug. 5, 1998, by Korzekwa et al., and U.S. patent application Ser. No. 09/811,283 “Predicting Metabolic Stability of Drug Molecules,” filed Mar. 15, 2001, by Ewing et al. Each of the above patent applications is incorporated herein by reference in its entirety and for all purposes.

### FIELD OF THE INVENTION

The present invention relates generally to systems and methods for analyzing the reactive sites of molecules, in particular drugs. More specifically, the invention relates to systems and methods for generating accessibility correction factors to electronic models of substrate metabolism, in particular substrates metabolized by the cytochrome P450 enzymes. These correction factors are used as part of the process to model and predict the metabolic properties of a substrate, as well as to engineer the substrates to achieve desired metabolic properties.

### BACKGROUND OF THE INVENTION

Bringing a single drug to market costs about $500 million to $1 billion dollars, with the development time being about 8 to 15 years. Drug development typically involves the identification of 1000 to 100,000 candidate compounds distributed across several compound classes that eventually lead, to a single or at most a few marketable drugs.

Those thousands of candidate compounds are screened against biochemical targets to assess whether they have the pharmacological properties that the researchers are seeking. This screening process leads to a much smaller number of “hits” (perhaps 500 or 1000) which bind with a target receptor and which are narrowed to even fewer “leads” perhaps 50 or 100) which appear most efficacious. At this point, typically, the lead compounds are assayed for their ADMET/PK (absorption, distribution, metabolism, elimination, and toxicity/pharmacokinetic) properties. They are tested using biochemical assays such as Human Serum Albumin binding, chemical assays such as pK_{A} and solubility testing, and in vitro biological assays such as metabolism by endoplasmic reticulum fractions of human liver, in order to estimate their actual in vivo ADMET/PK properties. Most of the lead compounds are discarded because of unacceptable ADMET/PK properties.

In addition, even optimized leads that have passed these tests and are submitted for FDA clinical trials as investigational new drugs (INDs) will often show undesirable ADMET/PK properties when actually tested in animals and humans. Abandonment or redesign of optimized leads at this stage is extremely costly, since FDA trials require formulation, manufacturing and extensive testing of the compounds.

The development of compounds with unacceptable ADMET/PK properties thus contributes greatly to the overall cost of drug development. If there was a process by which compounds could be discarded or redesigned at an earlier stage of development (the earlier the better), then great savings in terms of money and time could be achieved. The current tools essentially offer no comprehensive method by which this can be done.

A large portion of all drug metabolism in humans and most all higher organisms is carried out by the cytochrome P450 enzymes. The cytochrome P450 enzymes (CYP) are a superfamily of heme-containing enzymes that include more than 700 individual isozymes that exist in plant, bacterial and animal species. Nelson et al. Pharmacogenetics 1996 6, 1-42. They are monooxygenase enzymes. Wislocki et al., in Enzymatic Basis of Detoxification (Jakoby, Ed.), 135-83, Academic Press, New York, 1980. Although humans share the same several CYP enzymes, these enzymes can vary slightly between individuals (alleles) and the enzyme profile of individuals, in terms of the amount of each enzyme that is present, also varies to some degree.

It is estimated that in humans, 50% of all drugs are metabolized partly by the P450 enzymes, and 30% of drugs are metabolized primarily by these enzymes. The most important CYP enzymes in drug metabolism are the CYP3A4, CYP2D6 and CYP2C9 isozymes. While modeling techniques do exist for predicting substrate metabolism by enzymes other than CYP enzymes, no sufficiently accurate technique exists for modeling metabolism by the CYP enzymes. To the extent that modeling techniques are available for other enzymes, they work by analyzing either the interactions between enzyme and substrate, or the common characteristics for a series of substrates. See, for example, Schramm, “Enzymatic transition states and transition state analog design.” Annu Rev Biochem 1998; 67: 693-720; Hunter, “A structure-based approach to drug discovery; crystallography and implications for the development of antiparasite drugs.” Parasitology 1997; 114 Suppl: S17-29; Gschwend et al, “Molecular docking towards drug discovery.” Mol Recognit 1996 Mar-Apr; 9(2): 175-86.

While these modeling techniques are partially effective for some enzymes, they are frequently ineffective for the CYP enzymes. This is because the models give very heavy weight to the binding characteristics of the enzyme in question. For CYP enzymes, a substrate's “intrinsic” electronic reactivity matters more than its binding characteristics. CYP enzymes lack the high binding specificities that characterize most other enzymes. CYP3A is almost completely nonspecific from a binding perspective, while CYP2D6 and CYP2C9 are only modestly specific. Gross steric and electrostatic properties of a substrate have a secondary effect on their metabolism by the CYP enzymes.

Systems and methods that provide effective quantum mechanical and structural descriptor based models of substrate metabolism are disclosed in U.S. patent application No. 09/368,511, U.S. patent application No. 09/613,875, and U.S. patent application 09/811,283. While the effect of accessibility to a binding site is more limited with CYP enzymes than it is with other enzymes, accessibility still plays a role in substrate metabolism—particularly for some classes of substrate. Note that the potential advantage of accessibility adjustments to quantum mechanical modeling is discussed in, for example, Korzekwa et al, “Predicting the Cytochrome P450 Mediated Metabolism of Xenobiotics.” Pharmacogenetics (1993) v. 3, p. 1-18 and U.S. patent application 09/613,875.

In view of the foregoing, techniques for modeling accessibility effects, particularly in enzyme-substrate interactions such as interactions with CYP enzymes, in conjunction with quantum mechanical modeling of these interactions, would be highly beneficial.

### SUMMARY OF THE INVENTION

The present invention addresses this need by providing methods, programs and apparatus for generating accessibility correction factors. These factors may be used to modify values predicted by models of electronic component substrate reactivity. These correction factors can also be used to model other ADMET/PK properties where accessibility factors are important, such as absorption and toxicity.

In one aspect the invention, multiple separate correction factors generated in accordance with this invention are used to correct the electronic component of substrate reactivity. Most of the correction factors described herein pertain to either steric or orientation effects on substrate accessibility. Sometime a substrate will include portions or moieties that sterically hinder potential reaction sites, and thereby reduce the likelihood that a particular reaction site will actually react. The steric correction factors provide a measure of this steric hindrance. Sometimes a substrate will have potential reaction sites that cannot be oriented within or on a protein active site in a manner that allows reaction. This can occur because the overall shape or arrangement of physicochemical groups on the substrate molecule prevents smooth docking with the protein binding site. The orientation correction factors provide a measure of this orientation hindrance.

The correction factors used with this invention may be derived in many different ways. In one preferred embodiment, they are derived from one or more “descriptors” of the substrate structure. Each group of descriptors and associated correction factor pertain to a particular site on the substrate. Examples of such descriptors include site polarity, protrusion, partial surface area, partial charge, etc. Often the correction factor is a function of multiple descriptors. The function may be an expression comprising multiple terms, each representing the weighted contribution of a particular descriptor. In other embodiments, the correction factor is simply a descriptor or a descriptor multiplied by a coefficient or other function.

Generally, a model of this invention will predict the reactivity of a reaction site or the relative reactivity of a site in comparison to other sites on a given substrate. For each site on the substrate, the reactivity will have an electronic or intrinsic component and an accessibility component: E_{A}=E_{A0}+Accessibility Correction, where E_{A0} is the electronic component. The reactivity may take the form of an activation energy or rate constant, for example. As mentioned the accessibility correction may often have steric and orientation components. Thus, the model may be recast in a more detailed form:

$${E}_{{A }_{corr}} = {E}_{A , 0} + {\sum }_{l}^{N steric descriptors} {C}_{l} {K}_{i} + {\sum }_{j}^{M orientation descriptors} {C}_{j} {K}_{j} .$$

In this expression, the Cᵢs and Cⱼs are coefficients for the steric and orientation descriptors, respectively. And the Kᵢs and Kⱼs are the steric and orientation descriptors.

The Kᵢ are the steric accessibility descriptors, which include the surface area, parabolic curve, protrusion and extension descriptors. The Kⱼ are the orientation descriptors which may include distance-to-polar-regions, protrusion-weighted-distance-to-polar-regions, amphoteric moment, hydrophobicity, and distance-to-charged-atoms.

Another aspect of the invention pertains to methods of predicting the susceptibility of a reactive site on a molecule to metabolism. The method may be characterized by the following sequence: (a) receiving a value of an electronic contribution to reactivity for the site; (b) calculating an accessibility correction factor for the site; (c) applying the accessibility correction factor to the initial activation energy value to generate a new reactivity value for the site; and (d) outputting the new reactivity value for the site. Preferably, (a), (b), (c), and (d) are repeated for multiple reactive sites on the substrate molecule so that it can be determined which of the multiple reactive sites is most likely to undergo metabolism or to what extent the entire molecule is susceptible to metabolism.

Another aspect of the invention pertains to methods for calculating a steric accessibility correction factor, the method including generating steric accessibility descriptors for each reactive site, generating coefficients for each descriptor and outputting a steric accessibility correction factor for each site. Another aspect of the invention pertains to a similar method for calculating an orientation accessibility correction factor, except that orientation accessibility descriptors are used to generate the orientation accessibility correction factor.

Another aspect of the invention pertains to methods for calculating surface area steric effects on xenobiotic metabolism, the method including the operations of choosing a probe radius, determining the exposed surface area of an atom, comparing the exposed surface area to a reference value and outputting a surface area correction factor. The method is typically repeated for each reactive site of the molecule to generate correction factors for all the reactive sites.

Another aspect of the invention pertains to methods for calculating parabolic curvature steric effects on xenobiotic metabolism, the method including the operations of a point on or near one of the atoms, parameterizing at least one parabola using a point on or near an atom that is within about 10 Å of the atom in the reactive site and outputting a parabolic curvature correction factor. The method is typically repeated for each reactive site of the molecule to generate correction factors for all the reactive sites.

Another aspect of the invention pertains to methods for calculating protrusion steric effects on xenobiotic metabolism, the method including the operations of choosing an atom in the reactive site, extending a vector from a reference point in the molecule to the atom, assigning a score to the vector and outputting a protrusion correction factor. The method is typically repeated for each reactive site of the molecule to generate correction factors for all the reactive sites.

Another aspect of the invention pertains to methods for calculating extension steric effects on metabolism, the method including the operations of choosing an atom, extending a vector from the a reference point in the molecule to the atom, assigning a score to the vector and outputting an extension correction factor. The method is typically repeated for each reactive site of the molecule to generate correction factors for all the reactive sites.

Another aspect of the invention pertains to methods for calculating the location of polar regions orientation effects on metabolism, the method including the operations of calculating the polarity of each atom on the molecule, outputting a distance-to-polar-regions orientation accessibility correction factor. The method is typically repeated for each reactive site of the molecule to generate correction factors for all the reactive sites.

Another aspect of the invention pertains to methods for calculating amphoteric effects on xenobiotic metabolism, the method including the operations of calculating an amphoteric moment for the molecule, extending a vector from a reference point in the molecule to the atom, calculating a dot product of the amphoteric moment and the vector and outputting an amphoteric correction factor. The method is typically repeated for each reactive site of the molecule to generate correction factors for all the reactive sites.

Another aspect of the invention pertains to methods for calculating the hydrophobic character orientation effects on metabolism, the method including calculating the partial charge and partial surface area of all hydrogens connected to the reactive carbon, and outputting a hydrophobicity correction factor. The method is typically repeated for each reactive site of the molecule to generate correction factors for all reactive sites.

Another aspect of the invention pertains to methods for calculating proximity to charged atoms orientation effects of metabolism, the method including calculating the partial charge of each atom, calculating the distance from each atom to the reactive site, and outputting a proximity-to-charged-atoms correction factor. The method is typically repeated for each reactive site of the molecule to generate correction factors for all reactive sites.

Yet another aspect of the invention pertains to computer program products including machine-readable media on which are stored program instructions for implementing some portion of or all of a method as described above. Any of the methods of this invention may be represented, in whole or in part, as program instructions that can be provided on such computer readable media. In addition, the invention pertains to various combinations of data generated, stored, and/or used as described herein. The invention also pertains to apparatus on which the above methods may be performed, in whole or in part.

These and other features of the present invention will be described in more detail below in the detailed description of the invention and in conjunction with the following figures.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is a schematic illustration of the mammalian cyctochrome P450 catalytic cycle, including the non-metabolic decoupling reactions.

FIG. 2 is a schematic illustration of a substrate molecule (drug) with several reactive sites.

FIGS. 3A and 3B together make up a flowchart for determining the relative reaction rates of a substrate molecule, starting with the substrate's molecular structure.

FIG. 3C shows an anisole molecule, which has both an aliphatic and aromatic reactive sites.

FIG. 3D is a schematic illustration of a regioselectivity table that is generated to describe the relative rates of the reactive sites of a substrate molecule.

FIG. 3E is a schematic illustration of a relative rates curve plotted with the results from a regioselectivity table.

FIG. 4 is a high-level flowchart of one process for generating the accessibility correction factors and, from these, generating a corrected E_{A}.

FIG. 5 is a flowchart that illustrates a process for generating the surface area descriptor.

FIG. 6A is a flowchart that illustrates a process for generating the parabolic curvature descriptor.

FIG. 6B is a schematic illustration of how a parabola is generated for a reactive site on the molecule triazolam.

FIG. 7A is a flowchart that illustrates a process for generating the protrusion descriptor.

FIG. 7B is a schematic illustration of how the protrusion descriptor is generated for a reactive site.

FIG. 8 is a flowchart that illustrates a process for generating the amphoteric moment descriptor.

FIG. 9 is a flowchart that illustrates a general process for generating orientation accessibility descriptors.

FIG. 10 is a schematic illustration of a reactive site and several polar sites of a substrate.

FIG. 11 is a schematic illustration of how protrusion affects orientation accessibility.

FIG. 12 is a flowchart that illustrates a general process for generating steric accessibility descriptors.

FIG. 13 schematic illustration of how the extension descriptor is generated for a reactive site.

FIG. 14 is a flowchart depicting typical operations that may be employed to generate a model in accordance with an embodiment of this invention.

FIG. 15 is a representation of the relation between the atomic descriptor and the corrected energy values.

FIGS. 16A and 16B are flowcharts that illustrate one process for determining descriptor coefficients.

FIG. 17 illustrates a relative atomic stability plot.

FIGS. 18A and 18B illustrate a computer system suitable for implementing embodiments of the present invention.

FIG. 19 a schematic illustration of an Internet-based embodiment of the current invention.

### DETAILED DESCRIPTION

Introduction

In the following detailed description of the present invention, numerous specific embodiments are set forth in order to provide a thorough understanding of the invention. However, as will be apparent to those skilled in the art, the present invention may be practiced without these specific details or by using alternate elements or processes. In other instances well known processes, procedures and components have not been described in detail so as not to unnecessarily obscure aspects of the present invention.

Various scientific and technical terms are relevant to this invention and appear throughout the specification. To assist in understanding the terms and concepts presented herein, the following simple explanations are provided. The scope of the invention should not necessarily be limited by the following examples.

A “metabolic enzyme” is any enzyme that is involved in xenobiotic metabolism. Many metabolic enzymes are involved in the metabolism of exogenous compounds. Metabolic enzymes include enzymes that metabolize drugs, such as the CYP enzymes, uridine-diphosphate glucuronic acid glucuronyl transferases and glutathione transferases.

“Xenobiotic metabolism” is a term pertaining to any and all metabolism of foreign molecules that occurs in living organisms, including anabolic and catabolic metabolism.

A “reactive site” is a site on a substrate molecule that is susceptible to metabolism and/or catalysis by an enzyme. It is to be distinguished from a “active site,” which is the region of an enzyme that is involved in catalysis.

“Reaction rate” refers to the kinetic rate of a chemical reaction or a single step of a chemical reaction. The reaction rate can be predicted by modeling a compound's electronic reactivity and, in some cases, its interaction with a catalyst such as an enzyme. The electronic reactivity can be predicted from a transition state or by estimating the activation energy from the difference in free energy between a substrate and an intermediate form. The reaction rate can be predicted by descriptor based models of the substrate. The term “reaction velocity” is used interchangeably with “reaction rate.”

“Metabolism rate” refers to the overall rate of metabolism of a substrate, regardless of which reactive sites are involved in the metabolism of the drug to a non-reactive form. Thus the reaction rates of all of the reactive sites are involved in determining the metabolic rate.

“Activity” refers to an important characteristic of a compound. In a sense, an activity is like a “property” of a compound. However, in the context of this invention, activity usually refers to a biochemical, biological, and/or therapeutic behavior of a compound. Also, the activity of a compound is usually a characteristic that is to be predicted. Often, an activity serves as a dependent variable related to descriptors, which are independent variables. The models of this invention predict activity from descriptor values. Site specific reactivity of a substrate is an example of an activity predicted by this invention.

Depending on how a model is constructed, activity may take the form a specific numerical value (e.g., Eᵢ) or a threshold or filter (e.g., binds or does not bind).

A “complex” is an enzyme-substrate complex formed by covalent and other physicochemical bonds that may or may not lead to metabolism of the substrate/drug.

A “catalytic cycle” is a series of substrate reaction steps that are catalyzed or otherwise facilitated by an enzyme. One example described herein is the CYP catalytic cycle.

“Descriptor” refers to a variable or value representing a property of a particular compound. The property may pertain to the compound as a whole, a region or fragment of a compound, or individual atoms of the compound. Descriptors may be viewed as quantitative or textual representations of properties. They appear in expressions or models for predicting “activities” of a particular compound. A potentially infinite number of descriptors may characterize a compound. Multivariate models employ two or more descriptors to predict the activity of a compound.

“Accessibility” refers to the degree to which steric and orientation characteristics of a molecule affect its rate of metabolism and activation energy. “Accessibility correction factors” are factors that quantify these characteristics.

“Orientation accessibility” refers to the degree to which the orientation of a molecule with respect to an enzyme's active site affects the rate of metabolism and/or the activation energy of the molecule or a particular site on the molecule. “Orientation accessibility descriptors” are used to quantify these characteristics. Orientation accessibility descriptors are structural parameters that influence the ability of a molecule to orient itself on or within an enzyme's active site so that a reaction at a particular site is likely to proceed.

“Steric accessibility” refers to the degree to which the steric characteristics of a molecule affect the rate of metabolism and activation energy of the molecule or a particular site on the molecule. “Steric accessibility descriptors” are used to quantify these characteristics. Frequently, steric accessibility descriptors are chosen to characterize structural features that can hinder or block a region of a molecule (e.g., a reaction site) from cleanly contacting an active site of an enzyme. The hindering results from “crowding” by other moieties or regions on the molecule.

“Correction factor” refers to a variable or value that is used to correct activation energies or relative rates to account for the effects of steric and orientation accessibility. A correction factor may be a descriptor scaled by a coefficient in its simplest case, or it may be a combination of correction factors. In one example, the “amphoteric correction factor” is the “amphoteric descriptor” scaled by a coefficient, while the “orientation accessibility correction factor” is a linear combination of all the orientation accessibility correction factors.

A “Model” is a mathematical or logical representation of a physical and/or chemical relationship. Models may predict an activity from one or more descriptors of physical and/or chemical properties. In other words, such models treat an activity as a dependent variable and descriptors as independent variables. Thus, the model is itself a mathematical or logical relationship.

Models can take many different forms. They can take a very simple format such as a look up table or a more complex format such as a quantum chemical representation of an oxidation mechanism. Examples of the logical form of models include linear and non-linear mathematical expressions, look up tables, neural networks, and the like. In one preferred embodiment, the model form is a linear additive model in which the products of coefficients and transformed descriptors are summed. In another preferred embodiment, the model form is a non-linear product of various transformed descriptors (e.g., a multidimensional Gaussian expression).

Models can predict activity as a discrete event or a continuous range. A classification model predicts whether or not a discrete event such as binding will occur. Other models will predict the probability that the event will occur or the strength of the event (e.g., Kᵢ for enzyme substrate binding).

Models are typically developed from a training set of chemical compounds or other entities that provide a good representation of the underlying physical/chemical relationship to be modeled. The activities and descriptors form members of the training set and are used to develop the mathematical/logical relationship between activity and descriptors. This relationship is typically validated prior to use for predicting activity of new compounds.

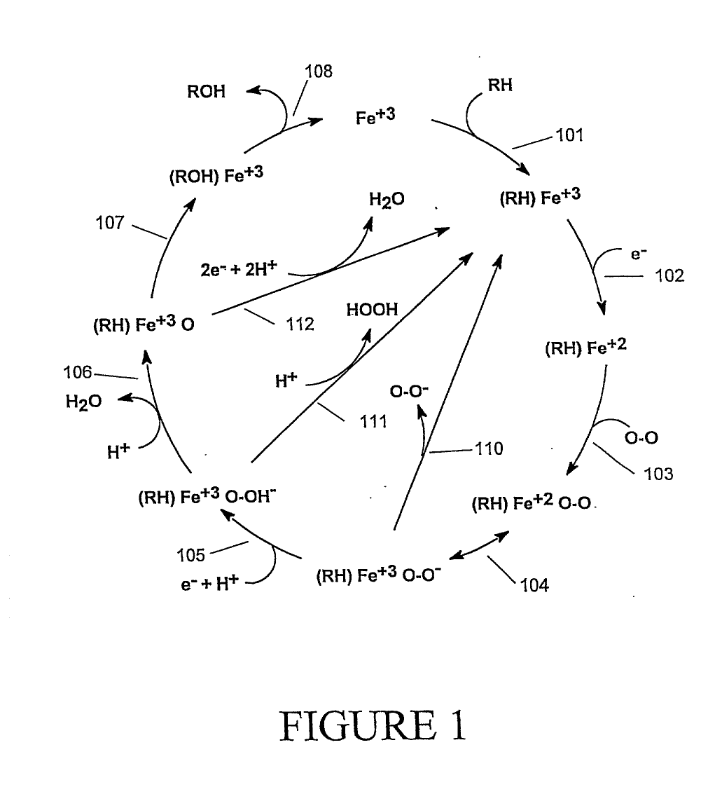

For background, FIG. 1 illustrates the oxidative hydroxylation catalytic cycle 100 for a mammalian CYP enzyme. The top of the figure shows a generic starting substrate (RH) and generic product (ROH). This hydroxylation reaction is often the first step in metabolizing an exogenous compound, and partly explains the importance of the CYP enzymes in drug deactivation/metabolism. Note that the hydroxylated product is not the only possible oxidation product produced by CYP enzymes; it is simply presented here for the sake of illustration. In addition, the described catalytic cycle is the generally accepted mechanism, but variations may occur between different P450 enzymes.

A first step 101 of the catalytic cycle 100 shows the initial binding of the substrate RH to the heme iron atom of the enzyme, which changes the equilibrium spin state of the heme iron from low to high. This lowers the reduction potential of the iron, thus facilitating transfer of an electron from NADPH, via cytochrome P450 reductase, to the iron atom in a second step 102. In a third step 103, molecular oxygen binds to the iron atom. In a fourth step 104, the bound oxygen is reduced by one electron and the iron is oxidized from a ferrous state to a ferric state. At this point, the oxygen can be decoupled from the enzyme as superoxide in a non-metabolic reaction, thus taking the enzyme-substrate complex back to its initial state (illustrated as the product of step 101) in a branch pathway step 110. Otherwise, the oxygen combines with one more electron and a proton in a fifth step 105, forming a peroxy intermediate with the enzyme-substrate complex. Here, a hydrogen peroxide decoupling reaction can take place, as illustrated in a branch pathway step 111, which takes the enzyme-substrate complex back to the initial state (again illustrated as the product of step 101).

Otherwise, in a sixth step 106, the peroxy intermediate reacts with another proton to undergo heterolytic cleavage, with one oxygen leaving the complex as a water molecule and the other oxygen coordinating with the iron atom as a reactive oxygen atom. A water decoupling reaction involving the addition of two protons and two electrons, illustrated as a branch pathway step 112, can take the enzyme-substrate complex back to the initial state. Otherwise, the reactive oxygen is transferred to the substrate to form an oxidized product (ROH), a seventh step 107. The product ROH then dissociates from the enzyme, an eighth step 108.

Note that the superoxide decoupling reaction 110, the hydrogen peroxide decoupling reaction 111, and the water decoupling reaction 112 all yield the substrate back in its original form in complex with the enzyme. These pathways thus reduce the rate of metabolism of the substrate. If either of the decoupling pathways predominate in the CYP catalytic cycle, then the substrate is unlikely to be metabolized rapidly.

Experimental evidence for the existence of these reaction pathways and intermediates is described in U.S. patent application No. 09/368,511, by Korzekwa et al. (Atty Docket No.: CAMIP001). That patent application also contains additional material on the mechanisms of CYP enzyme-substrate interactions.

This evidence also shows that the last steps of the CYP catalytic cycle, steps 107 and 108, are not typically the rate-limiting steps in the sense that they are not the slowest steps in the catalytic cycle. They are often the “product-determining” steps, however. While rate-limiting steps are usually thought of as the steps that determine the rate of product formation, if there is an alternate pathway that competes with a fast product formation step, that alternative pathway can unmask the rate of product formation.

Therefore the relative rates analysis of the present invention, while it applies to these last steps in the catalytic cycle, does provide useful, and often the most important, reaction rate information on substrate metabolism. To determine complete and absolute rates of substrate metabolism, at least some of the other reaction rates of in the CYP catalytic cycle may be considered. In a preferred embodiment, the model also accounts for either or both of the decoupling reactions 110 and 111. It appears that the peroxide decoupling step 111, for example, is somewhat substrate dependent. Therefore, the model may make use of certain substrate characteristics to predict the degree to which this decoupling reaction affects the absolute rate of metabolism.

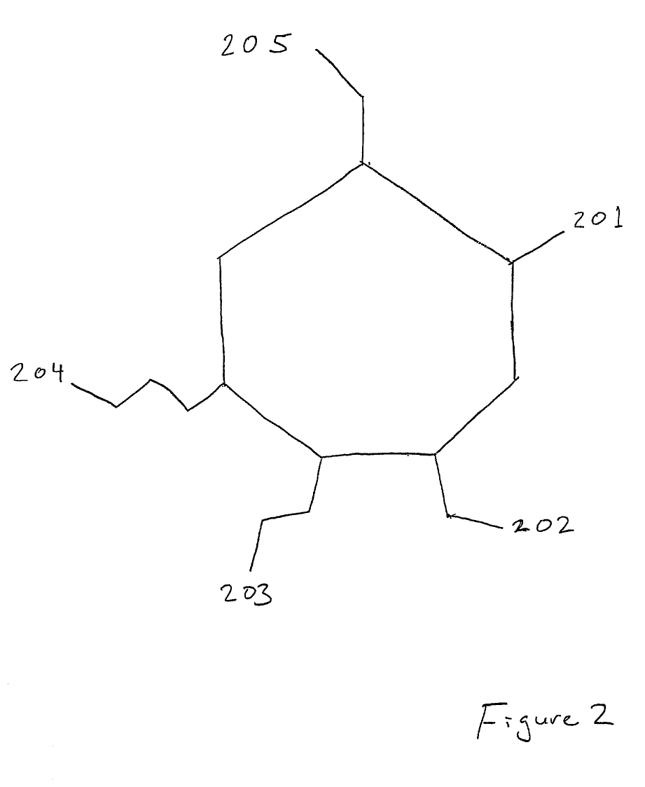

FIG. 2 is a simplified, schematic illustration of a substrate molecule with several reactive sites, 201-205, for CYP enzyme metabolism. Each of these sites may serve as the predominant oxidation site for CYP metabolism. Each of these sites may also be subject to one of the decoupling reactions set forth in FIG. 1. In each case, the probability that the site will react during metabolism is a function of the site's intrinsic reactivity in the enzyme's active site, the accessibility of the site to the enzyme's active site, and the relative rate of the corresponding decoupling reactions.

One of the most common ADMET/PK problems with a drug candidate is that it is metabolized too quickly. In many cases, an ideal drug would be metabolized slowly enough so that it can be administered about once a day. In the current art, if a drug candidate is being metabolized too quickly for daily administration, the designers of the drug will try to redesign it, typically by modifying the most reactive site in a manner that would make it much considerably more stable.

However, changing this most reactive site, even by making it extremely stable or even non-reactive, may or may not result in an appreciable decrease in the rate of metabolism of the drug. The result is essentially unpredictable by methods of the current art. A drug designer much less has the ability to predict how a more minor change in a reactive site will affect the metabolism of the drug. For instance, site 203 might be observed to be the most reactive site. A drug designer could then modify it to make more stable or even unreactive in an attempt to decrease the overall metabolic rate of the substrate. In some instances this will be successful, but if the substrate has one or more reactive sites that also have relatively high reactive rates, then these sites will often “take over” the metabolism of the substrate and the overall metabolic rate will remain essentially unchanged.

Therefore, a drug designer would have to go through the time-consuming process of redesigning one site as essentially a shot in the dark, re-testing the ADMET/PK properties, and then redesigning that site and/or one or more of the other reactive sites as additional shots in the dark. After conducting this process on most or all of the reactive sites of the drug, the designer might find that it is essentially impossible to achieve the ADMET/PK properties that are desired, particularly without reducing, or perhaps destroying, the desired pharmacological properties of the drug. The chances of altering the pharmacological properties of the drug greatly increase as more and more redesigns of the drug are carried out.

Slowing down the rate of metabolism of a drug candidate is by no means the only ADMET/PK property that drug designers try to affect. They also may try to speed up the rate of metabolism of drug. In addition, it is generally preferable that a drug have more than one deactivating pathway and/or reactive site, so that chances of dangerous drug interaction, via blocking the primary metabolic pathway, are minimized. The CYP enzymes are also susceptible to induction, so that one drug may induce faster metabolism of another drug. The fact that multiple reactive sites are often desirable, for both these reasons, can make the design of the drug even more complicated.

### Electronic Models and Accessibility Corrections

As mentioned, models of this invention generally predict the reactivity of a reaction site or the relative reactivity of a site in comparison to other sites on a given substrate. In this manner, the model can predict the likelihood that any given site on a substrate will contribute to the metabolism of that substrate.

For each site on the substrate, the reactivity will have an electronic or intrinsic component and an accessibility component: E_{A}=E_{A0}+Accessibility Correction, where E_{A0} is the electronic component. The reactivity may take the form of an activation energy or rate constant, for example. E_{A0} may be calculated in any of a number of ways. Often, though not necessarily, quantum mechanical models (ab initio and/or empirical versions) will provide value of the electronic component. Other types of models such as structural descriptor based models (atom, site, or fragment level descriptors), Hammett-type Linear Free Energy Models, physicochemical property based models, and the like may be used for this purpose. In each case, the model accounts for electronic contribution of site reactivity, wholly or partially unencumbered by accessibility criteria.

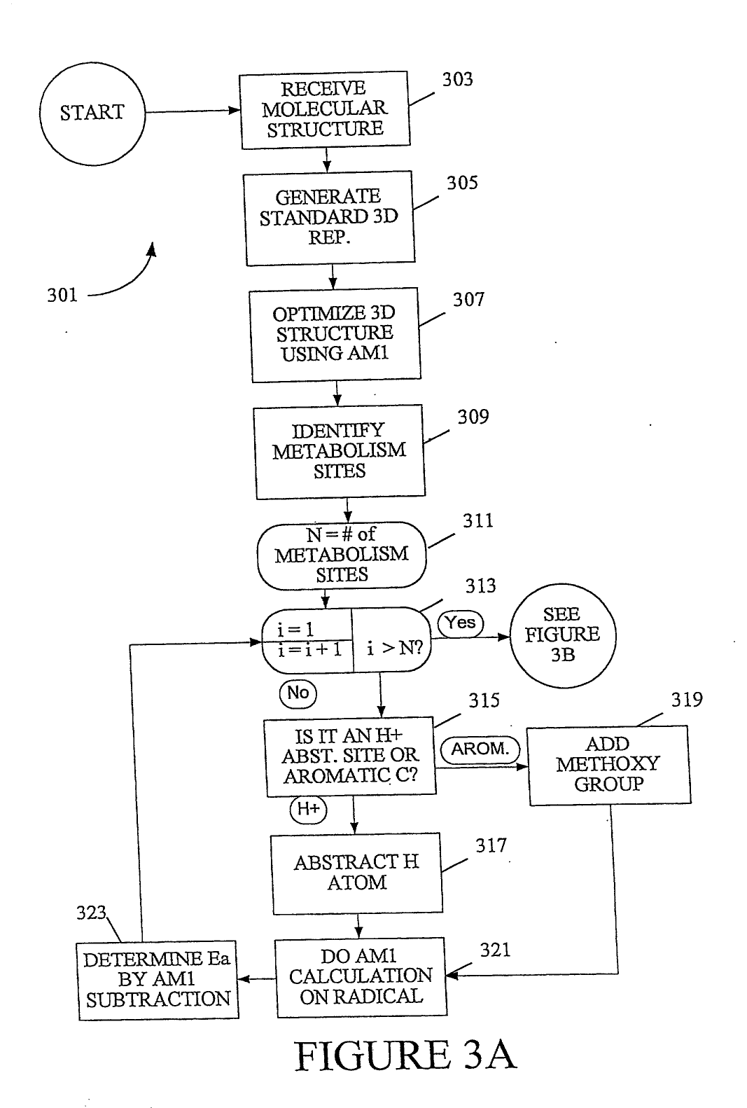

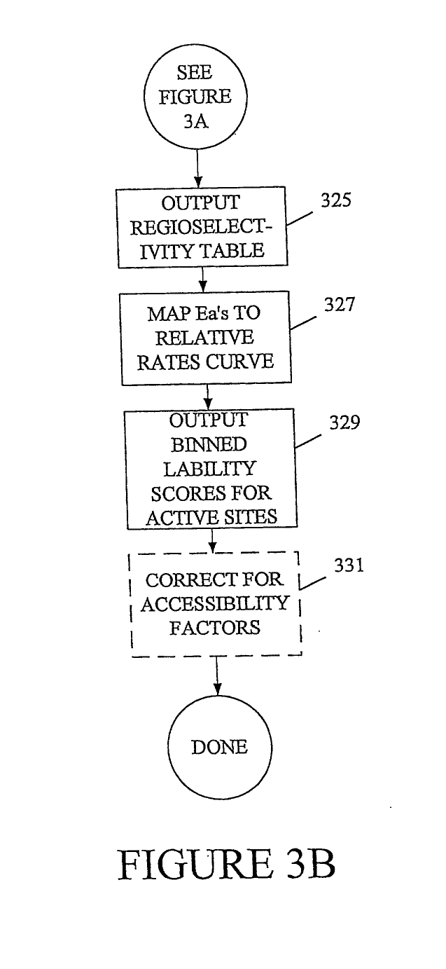

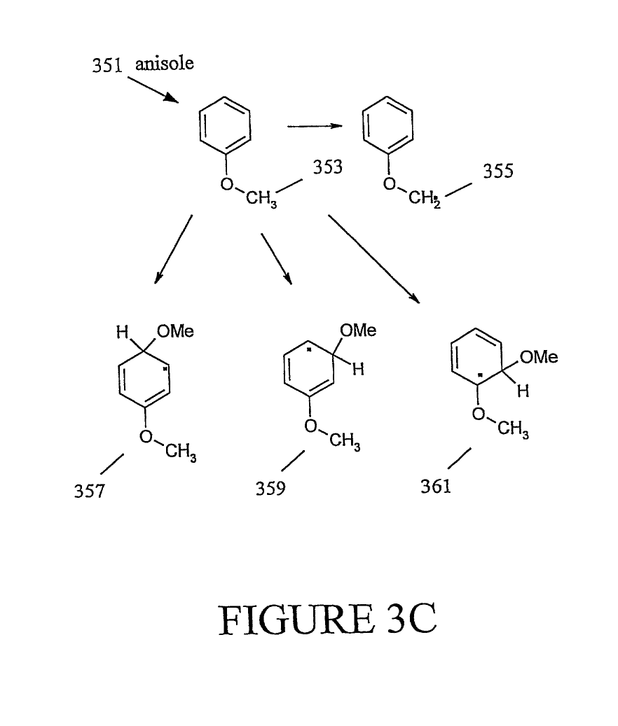

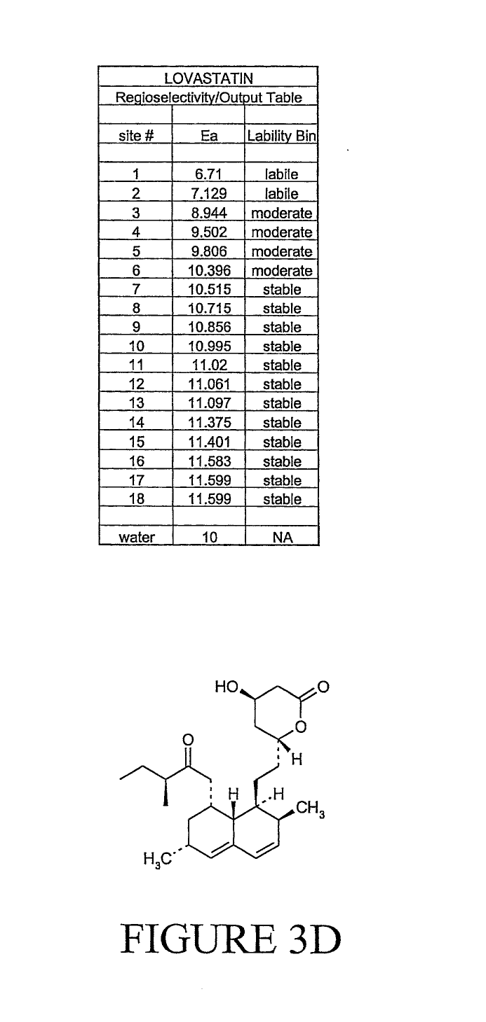

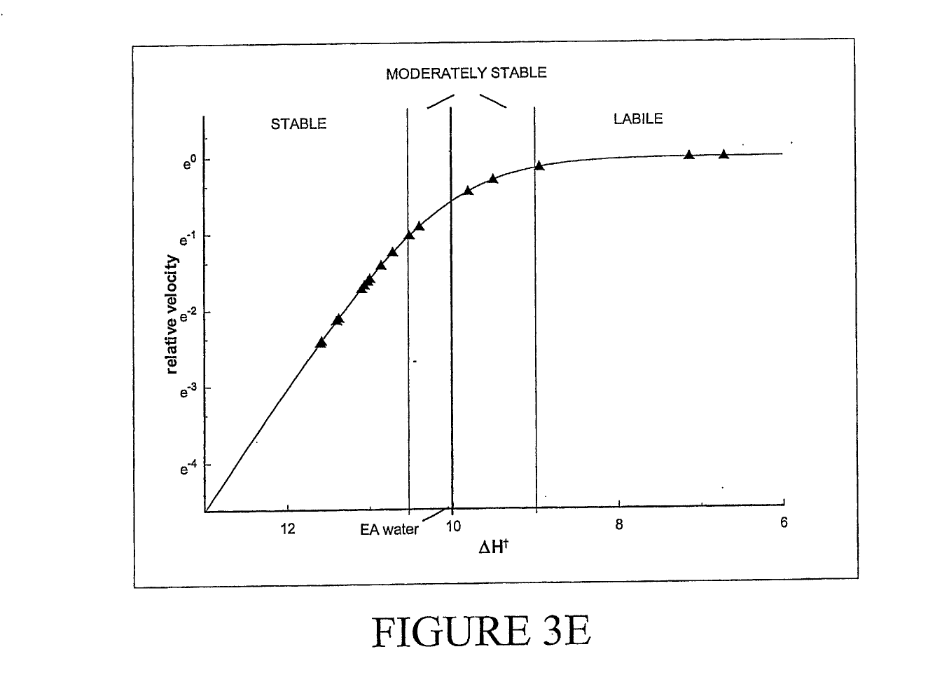

Various types of accessibility correction factors will be set forth below. At this point, an example of an overall model for predicting both the electronic and accessibility components of site-specific reactivity will be described. FIGS. 3A-3E illustrate the example. They are applicable to the present invention, but are not the only means of predicting substrate reactivity. Note that the specific models depicted employ quantum mechanical techniques for predicting the intrinsic reactivity of a substrate. Other models such as structural descriptor based models of the type described in U.S. patent application No. 09/811,283 may also be employed to predict intrinsic reactivity. In any case, the intrinsic reactivity is corrected using one or more accessibility correction factors of this invention.

FIGS. 3A and 3B together make up a flowchart illustrating from a high-level one preferred process, 301, for generating the relative rates curve and associated information for a substrate molecule. Initially at operation 303, the molecular structure of the substrate is received. The molecular structure can be received as an organic chemistry string of atoms, a two-dimensional structure, a IUPAC standard name, a 3D coordinate map, or as any other commonly used representation. If not already in 3D form, a 3D coordinate map of the molecule is generated, using a geometry program such as Corina or Concord. See 303. The 3D structure generator Corina is available from Molecular Simulations, Inc., of San Diego, Calif. and Molecular Networks GmbH of Erlange, Germany. Concord is available from Tripos, Inc. of St. Louis, Mo. Corina uses straightforward rules about molecular bond and functional group conformation to generate an approximate geometry 3D structure, which is optimized to a local energy minimum. For instance, if an amine group is encountered, then it will be placed in a planar conformation, as that group normally exists. Concord applies a similar method, but also uses a limited set of molecular mechanical rules involving branch angles, strain and torsion, to achieve its 3D structure.

This approximate 3D geometry structure is then optimized with a more sophisticated modeling tool, typically AM1. AM1 is a semi-empirical quantum-chemical modeling program that optimizes the given 3D structure to that local energy minimum. See 307. It calculates electron density distributions from approximate molecular orbitals. It also calculates an enthalpy value for the molecule. AM1 is available as part of the public-domain software package MOPAC, which is available from the Quantum Chemistry Program Exchange, Department of Chemistry, Indiana University, Bloomington, Ind. The MOPAC-2000 version of MOPAC can be obtained from Schrödinger, Inc., of Portland, Oreg.

The process then identifies each reactive site of metabolism on the molecule. See 309. In the preferred embodiment, the reactive sites include alkyl carbons and aromatic carbons. These sites are chosen because CYP enzymes generally oxidize the substrate molecules at these sites. Other reactive sites can be considered in other embodiments, depending on the enzyme and/or class of substrates under consideration. Examples of functional groups, susceptible to oxidation, that may be analyzed using the present invention include C—H, C—C, C≡C, C═C, C═O, C—N, C═N, —S—, —N—, —N═, —CHO, —OH, and —C—OH.

The process analyzes each reactive site, beginning with operations 311 and 313, where the system sets a variable N equal to the number of reactive sites to be considered (311) and iterates over those sites (311). Iterative loop operation 313 initially sets an index value “i” equal to 1. It then determines whether the current value of i is greater than the value of N. If not, it performs various operations to determine the activation energy (E_{A}) at that site.

In operation 315, the process determines whether the reactive site is an alkyl carbon or aromatic carbon site. If it is an alkyl carbon site, the process will remove a hydrogen atom, in silico, from the site. See 317. The molecule in this state is an intermediate form of the molecule, which can be used to approximate the transition state the molecule will go in the oxidation reaction of step 108. The process then does a new AM1 calculation on the intermediate molecule to determine its 3D map and enthalpy. See 321. Note that the base molecule's 3D map and enthalpy were calculated at 307. The process then determines the enthalpy difference between the intermediate and base form of the molecule. Assuming that ΔS is close to zero, which is a good assumption for the conditions under which CYP oxidation takes place, the process yields a good approximation of the activation energy value (E_{A}) for the reactive site. Other properties of the radical, such as its ionization potential, can also be used in estimating the E_{A}. If the reactive site is an aromatic carbon, then the process will add a methoxy group to the molecule to form the intermediate-radical. See 319. The operations for doing a new AM1 calculation, 321, and determining the E_{A}, 323, are the same as they are for proton abstraction sites.

FIG. 3C shows an anisole molecule, 351, which has both an aliphatic and aromatic reaction sites and can be used to illustrated both hydrogen abstraction and methoxy addition. The aliphatic reaction site of the anisole is the terminal methyl group 353. When a hydrogen ion (proton) is abstracted from this group, the intermediate that results has an extra electron on the reactive carbon. See 355. The aromatic ring can react in an ortho, meta or para fashion, with the methoxy group adding to those position as shown in intermediates 357, 359 and 361, respectively. The addition leaves a free electron on the ring.

When i is greater than N, indicating that all the reactive sites have been analyzed, the process outputs a regioselectivity table or other arrangement of data that indicates the relative lability and activation energies of each of the reactive sites. See 325. A schematic example of such a regioselectively table is illustrated in FIG. 3D. The activation energies are used to map the reactive sites to a relative rates curve. See 327. A schematic example of such a relative rates curve is shown in FIG. 3E. The reactive sites are then binned based upon their relative rates. See 329. The reactive sites are typically binned into three categories: labile, moderately labile and stabile.

This concept of lability is typically specified with reference to a decoupling pathway in the enzyme's catalytic cycle. In the case of the CYP enzymes, the decoupling pathways are illustrated as steps 110, 111 and 112, which are the superoxide, hydrogen peroxide and water decoupling pathways. This is because these decoupling pathways regenerate the unreacted substrate. Substrate reactions with metabolic pathways that compete with, and proceed more rapidly than, these decoupling reactions provide for significantly faster metabolism. The relative rates data of the preferred embodiment specifically applies most directly to the last metabolic steps of the CYP catalytic cycle, steps 107 and 108, as they compare with the rate of water decoupling.

The final operation is the accessibility correction operation. See 331. As stated earlier, the CYP enzymes, particularly 3A4, do not have the same binding specificities that other enzymes are. However, in certain cases, a reactive site may be deeply buried within the substrate molecule, or the molecule may have a strongly preferred binding orientation, so that the relative rate of the reactive site is hindered or accelerated. In such cases, the user may wish to incorporate accessibility correction factors, as described below. Also, when the accessibility corrections factor of operation 331 are calculated, it is necessary to repeat operations 325 to 331 for outputting regioselectivity tables and rate curves. In a preferred embodiment, operation 325 to 331 are often delayed until after operation 331, to avoid outputting the data twice.

In any case, it is worth noting that the core process for determining the relative rates data and the steric corrections is generally carried out without reference to the drug metabolizing CYP enzymes. As long as the enzymes being studied carry out metabolism by similar mechanisms, the data from one analysis can usefully be applied to many enzymes. The orientation corrections often apply specifically to only a subset of the CYP enzymes for several reasons. CYP3A4 is recognized as the primary metabolizing enzyme for generally hydrophobic xenobiotics. Although the three-dimensional structure of the human CYP enzymes is currently unknown, it is thought that the active site of CYP3A4 must be generally hydrophobic and flexible in order to efficiently metabolize both small and very large compounds. It has been observed that adding a strongly polar group to a hydrophobic compound tends to reduce metabolism at nearby sites and increase metabolism at distal sites, implying that the active site of CYP3A4 may contain a hydrophilic region. CYP2D6 has a substrate selectively for positively charged compounds leading researchers to propose that negatively charged regions must exist in the CYP2D6 active site. CYP2C9 has a substrate selectivity for compounds with electronegative as well as aromatic functional groups, implying the possible presence of aromatic and electropositive regions in the CYP2C9 active site.

### DESCRIPTORS AND CORRECTION FACTORS

As mentioned above, the correction factors of this invention may be obtained by various methodologies and expressions. The degree of complexity and range of descriptors can vary widely from factor to factor. In the subsequent discussion, a relatively simple set of correction factors will be described first. This set includes only a single descriptor for each correction factor. Later, a more detailed set of correction factors will be described. Each includes a summation of multiple terms. Each term is a product a coefficient and one or more descriptors.

In the simpler model, the corrected value of E_{A} is calculated by the following formula:

E_{A(new)}=E_{A(original)}+f_{SA}(K_{SA})+C_{P}K_{P}+C_{R}K_{R}+C_{A}K_{A}.

Here E_{A(new)} is the corrected value of activation energy, E_{A(original)} is the electronic component of activation energy, K_{SA} is a descriptor for surface area at the site, K_{P} is a descriptor for a parabolic curve at the reaction site, K_{R} is a descriptor for protrusion at the reactive site, and K_{A} is a descriptor for an amphoteric moment at the reaction site. The C values are coefficients for the respective descriptors. The f_{SA} is a function that modifies the descriptor for surface area.

In the more complex model, the expression for corrected activation energy may take the following form:

$${E}_{{A}_{corr}} = {E}_{A , 0} + {\sum }_{l}^{N steric descriptors} {C}_{i} {K}_{i} + {\sum }_{J}^{M orientation descriptors} {C}_{j} {K}_{J} .$$

In this expression, the Cᵢs and Cⱼs are coefficients for the steric and orientation descriptors, respectively. And the Kᵢs and Kⱼs are the steric and orientation descriptors.

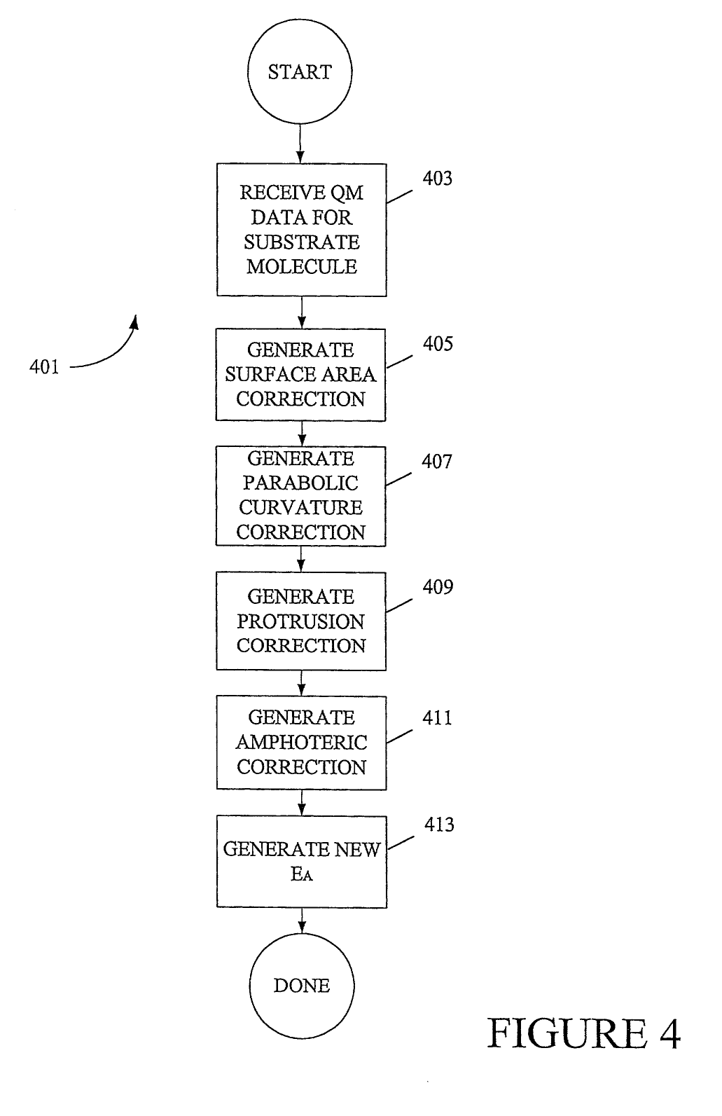

FIG. 4. illustrates from a high-level a process 401 for generating and applying the accessibility correction factors for the simpler model. In block 403, the intrinsic reaction rate data for a single reactive site, or more likely for an entire substrate molecule or set of substrate molecules is received. It is not absolutely necessary to have final calculations for the activation energies (E_{A}'s) at this point, since the correction factors are typically calculated as constant correction factors, though it is of course necessary to have the intrinsic reaction rate data in order to apply the correction factors to the E_{A}'S and calculate new E_{A}'S. It is necessary to have the substrate molecule's 3D coordinate map as calculated by AM1 and the reactive sites identified, however, and this information will typically be received together with the E_{A}'s.

In one example, the reactive sites include alkyl carbons and aromatic carbons. As mentioned, CYP enzymes generally oxidize the substrate molecules at these sites. Other reactive sites such as sulfur or nitrogen containing sites can be considered in other embodiments, depending on the enzyme and/or class of substrates under consideration. In a preferred embodiment, the process determines one type of descriptor for every site in the molecule before moving on to the other descriptors.

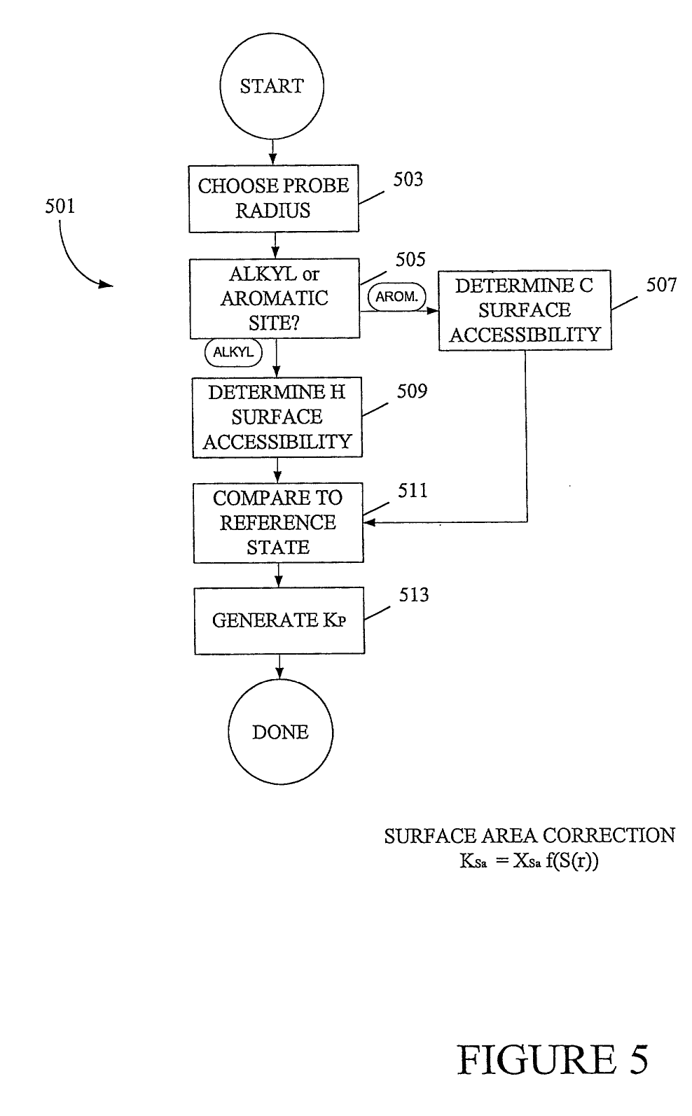

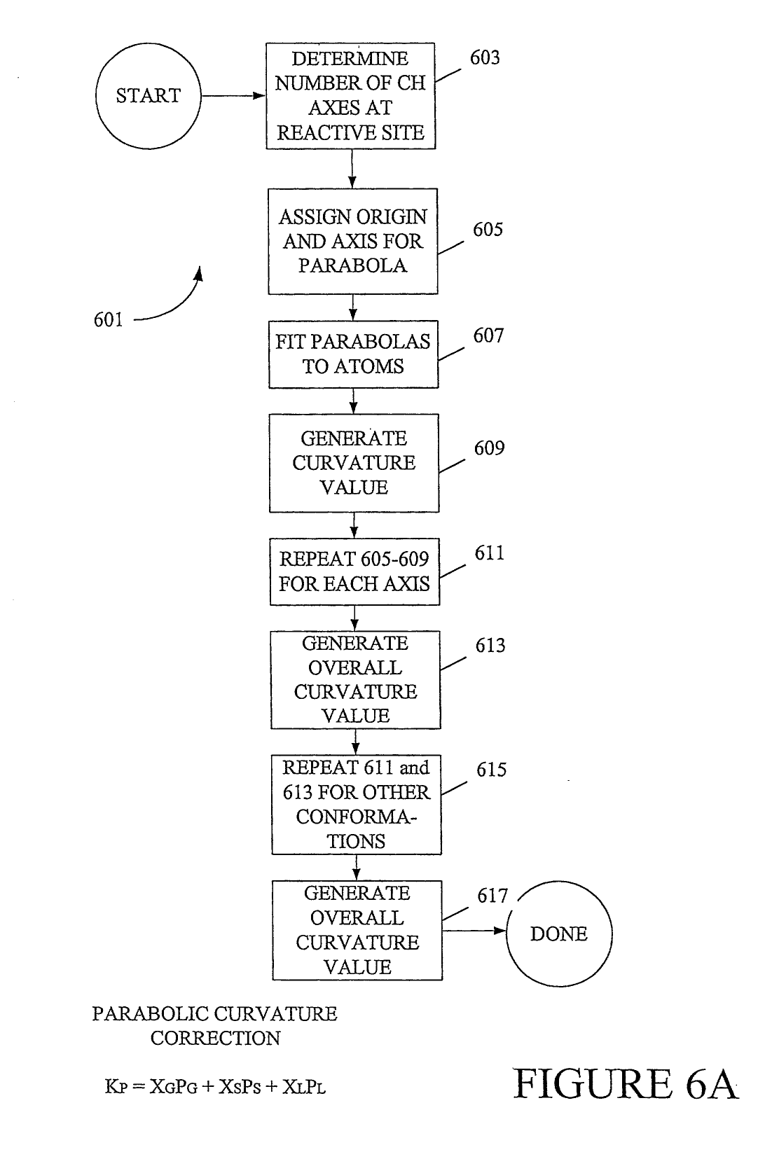

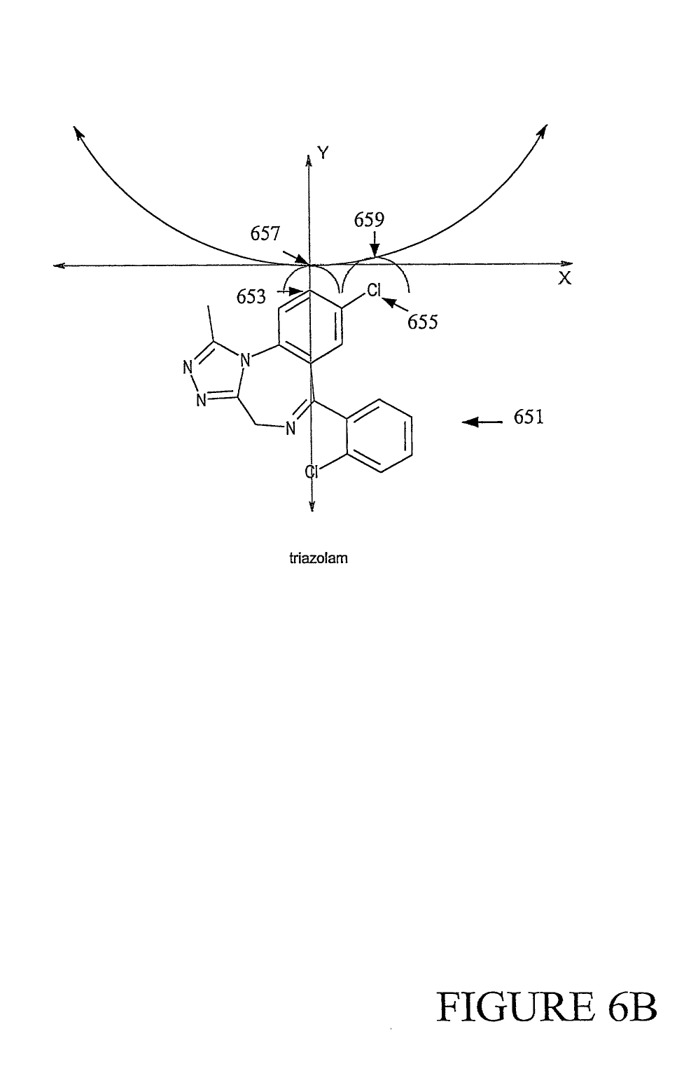

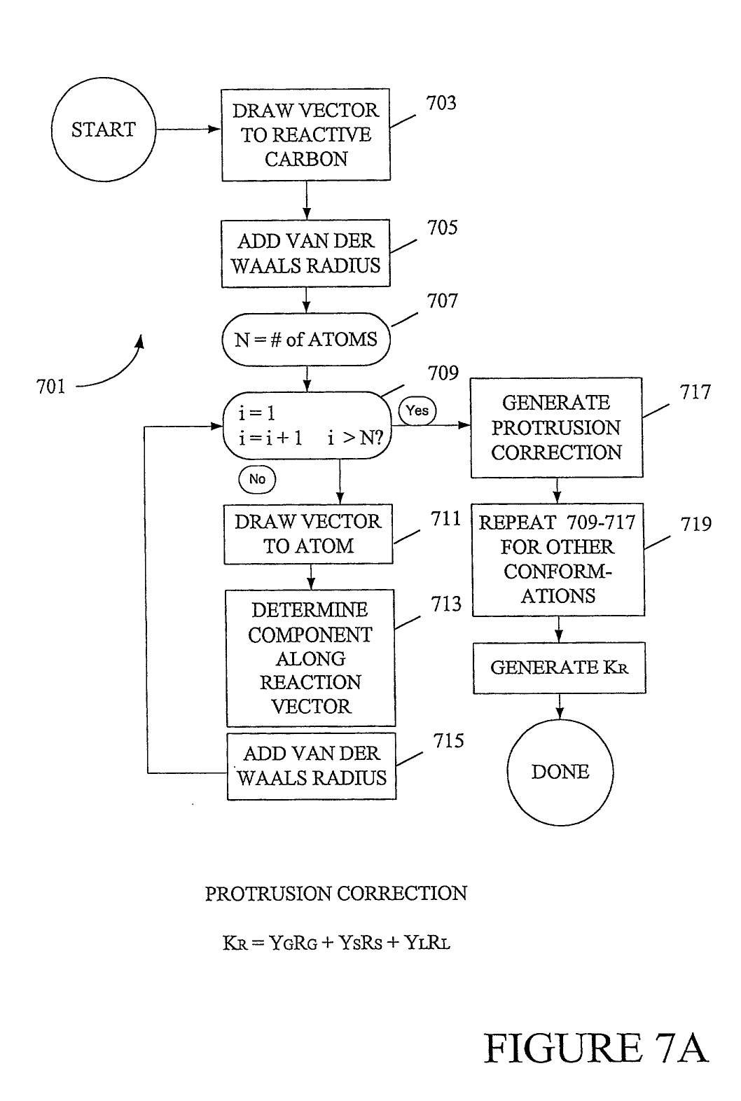

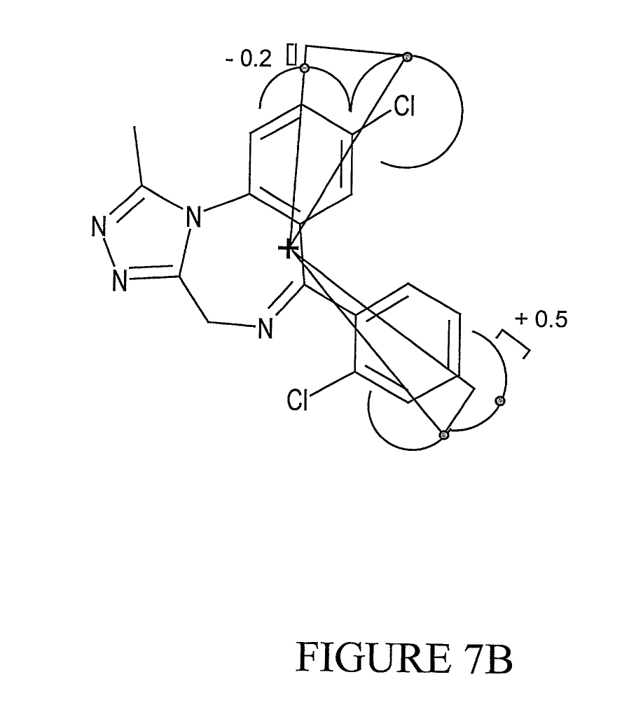

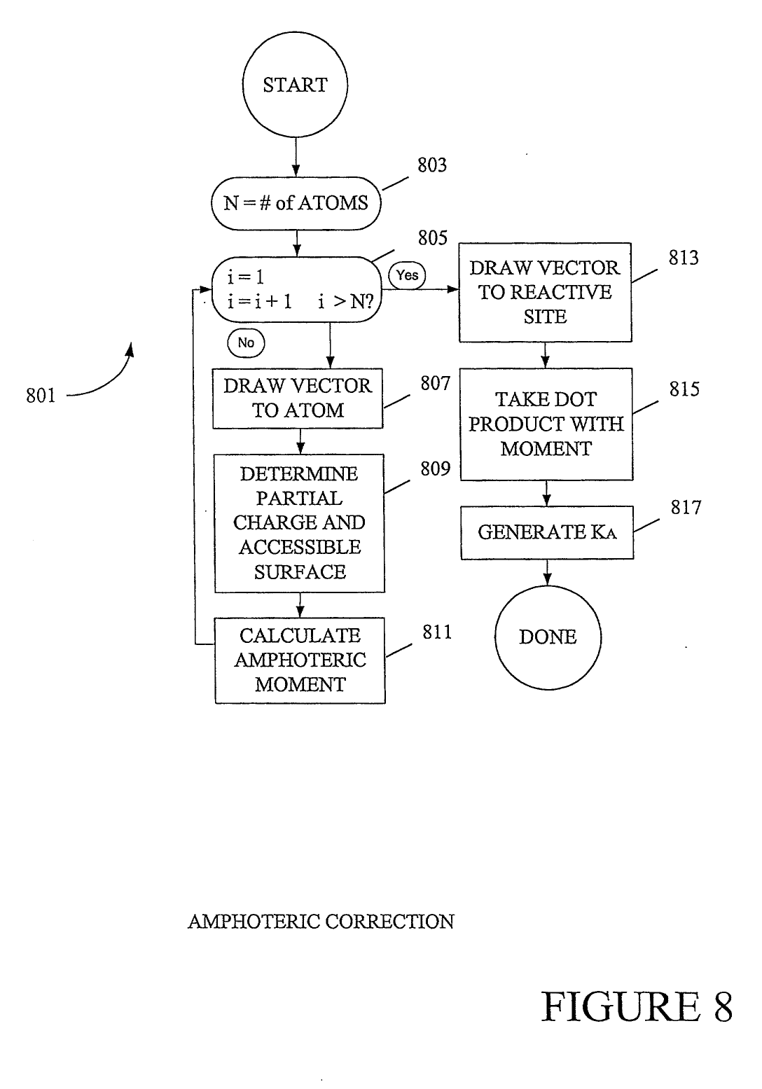

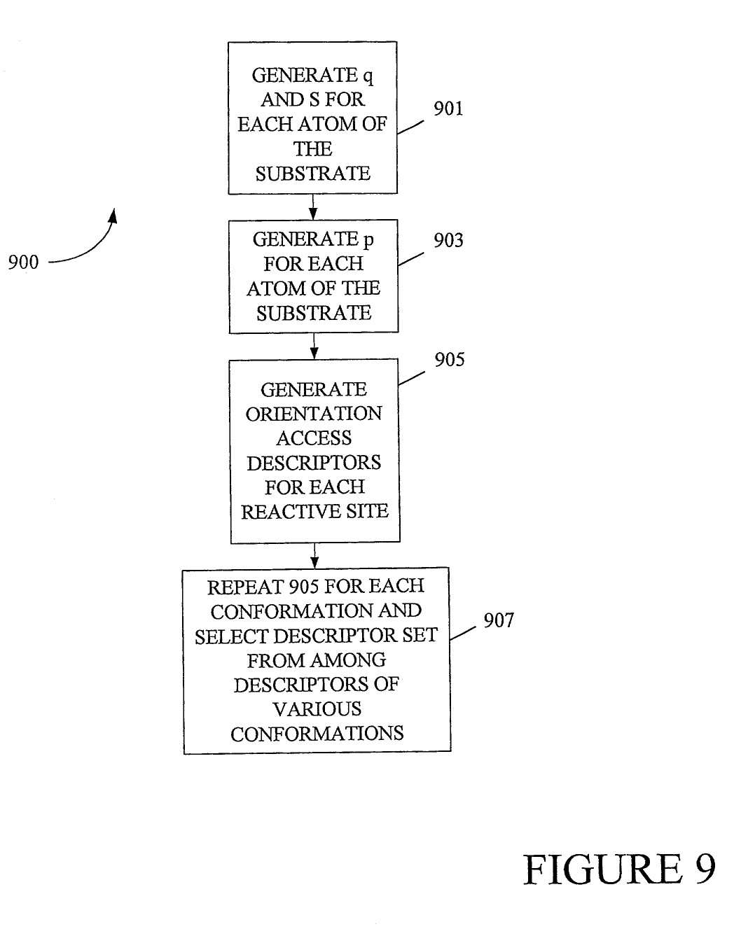

In operation 405, the first steric accessibility descriptor, surface area, is calculated. This operation is described in more detail with reference to FIG. 5. In block 407, the second steric accessibility descriptor parabolic curvature, is calculated. This is described in detail with reference to FIG. 6. In operation 409, the third steric accessibility descriptor protrusion, is calculated. This is described in detail with reference to FIGS. 7A and 7B. In operation 410, the fourth steric accessibility descriptor, extension is calculated. This is described in detail with reference to FIGS. 8A and 8B. In operation 411, the orientation accessibility descriptors are calculated. This is described in detail with reference FIG. 9. After all the descriptors are determined, correction factors are generated in operation 412. The correction factors are used to calculate the new E_{A} for the reactive site. See 413.

Again, the expression for the new activation energy has the following form:

E_{A(new)}=E_{A(original)}+f_{Sa}(K_{SA})+C_{P}K_{P}+C_{R}K_{R}+C_{A}K_{A}.

The second and third terms on the right side of the equation are the steric accessibility correction factor and the orientation accessibility correction factor, respectively. The correction factors are in the same units as the activation energies, and express a positive or negative additive correction to the original E_{A}. The fₛₐ(Kₛₐ) is a simple relative contribution/scaling function. The other can also be scaled with such a function, but in a preferred embodiment, they are scaled with the linear constants. In a preferred embodiment, f_{Sa}(K_{Sa}) is about −1n(Kₛₐ), C_{P} is about 8 to 10, C_{R} is about 0 to 1 and C_{A} is about 0 to 0.5. Including the scaling functions/constants, it has been found that the energies contributed by the correction processes to the new E_{A} are typically about 0 to 5 Kcal/mole for surface area, 0 to 5 Kcal/mole for parabolic curvature, −1 to 1 Kcal/mole for protrusion and −0.2 to 0.2 Kcal/mole for amphotericity. For strongly amphoteric molecules, values of about −2.0 to 2.0 Kcal/mole for amphotericity are typical.

FIG. 5 illustrates a preferred process for generating the surface area descriptor K_{SA}. See 501. Surface area accessibility is the amount of surface area of the reactive atom that is exposed on the surface of the substrate (as compared to chosen reference atom environments). As most actual atoms will be somewhat hidden as compared to the reference atoms, this factor will typically impose an energy penalty on the uncorrected electronic E_{A} for the reactive site. The calculated K_{SA} will typically have a value of 0 to 5 Kcal/mole.

In order to calculate the function K_{SA}=f(S(r)), the process chooses a probe radius r, which is typically the radius of the solvent molecule, usually water (about 1.4 Å) or larger solute (about 1.4 to 5 Å). See 503. The process then calculates an accessible atom surface based on this probe radius. See 505. If the reactive site is aliphatic, then the atom is the reactive hydrogen. See 507. If the reactive site is aromatic, then the atom is the reactive carbon. See 509. The accessible surface is then compared to a reference state, which is a hydrogen in a methyl group at the end of a long aliphatic chain for aliphatics, or a carbon at the para position in an aromatic ring for aromatics. See 511. This, along with simple modifying constants or functions, yields the final K_{SA}=f(S(r)). See 513. A simpler method of calculating the surface area of the atom using only the van der Waals radius of the atom may be used.

FIG. 6A illustrates a preferred process for generating the parabolic curvature descriptor K_{P}. See 601. Parabolic curvature is the influence of the shape of the reactive site on its reactivity. If the site is convex on the surface, then the site will be more reactive and the correction factor will decrease the E_{A} given by the electronic model. If the site is concave on the surface, then the site will be less reactive and the correction factor will increase the E_{A}. In this preferred embodiment, two-dimensional parabolas are used to estimate the three-dimensional paraboloid of the reactive site. Three-dimensional paraboloids, while more complex, can be used instead.

The process first determines the number of CH axes at the reactive site. See 603. For each CH axis, the process orients the molecule on a Cartesian plane according to the vector from the carbon to the hydrogen. See 605. The CH vector of the molecule determines the Y-axis, and the origin of the Cartesian plane is set at the van der Waals radius of the reactive carbon. FIG. 6B illustrates a triazolam molecule, 651, oriented according to this process, with the reactive site being the carbon, 653, ortho to the chlorine atom, 655. Next, all the atoms within a certain distance of the origin (typically 5 to 7 Å), are used to generate constant values according to the general parabolic equation y=c x². See 607. The (x,y) point for each atom is set at the van der Waals radius of the atom, in the direction of the CH vector, in order to calculate the constant c. From this set of parabola constants, an overall curvature value is calculated. See 609. In a preferred embodiment, this value is the Max value of all the constants.

FIG. 6B shows the parabola generated by using the chlorine atom. See 657. The (x,y) point of the chlorine atom is also shown. See 659. The parabola is slightly concave, and since the chlorine atom defines the Max concavity for this CH axis, the parabola indicates that the sight is slightly inaccessible due to its concavity, and that a positive K_{P} correction factor will result.

If there is more than one CH axis at the reactive site, operations 605-609 are repeated for each of them. Once curvature values for all the axes are calculated at operation 611, another overall curvature value is calculated. See 613. In a preferred embodiment, this value is again the Max value. This is the global parabolic curvature value, which itself can be used to calculated the parabolic descriptor K_{P}. In a preferred embodiment, the process also derives a local and semi-local curvature value. See 615. The local value is derived in the same manner at the global value, except that the atoms used are only those atoms within the chosen distance and that are conformationally rigid with respect to the origin. The semi-local value is also similarly derived, except that the atoms used are those within a chosen distance and that lie one rotatable bond away from the origin.

Many techniques exist to search the conformations accessible to flexible molecules. For highly flexible molecules, finding all the accessible low-energy conformations can become computationally intractable. It has been found that a modified systematic search algorithm can be useful to this conformational analysis, as well as to the protrusion correction factor analysis, which is described below. Rather than search all rotatable bonds simultaneously in a single search, multiple searches can be carried out in which rotatable bond subsets are processed. The subsets are selected based on mutual adjacency. Any two rotatable bonds are considered adjacent if they are separated by only non-rotatable bonds in the molecule connectivity graph. All possible subsets of adjacent rotatable bonds up a specified number, typically five rotatable bonds, are then enumerated. The advantage of considering adjacent rotatable bonds is that cooperativity effects can be better addressed. For linear extended molecules, this technique can quickly generate compact folded conformations. For branched molecules this technique is also useful, since the movement of one branch can significantly affect on the accessible space of another branch.

The process now has all the values needed to derive the parabolic curvature descriptor, K_{P}, where K_{P}=X_{G}P_{G}+X_{S}P_{S}+X_{L}P_{L}. See 617. The overall global, semi-local and local parabolic curvature values are G, S and L respectively. The relative contributions of these values are modified by the linear scaling/contribution constants X_{G}, X_{S} and X_{L}. In a preferred embodiment, X_{G} is 1.0 and the other modifying constants are zero. Typical K_{P} values obtained are −0.4 Kcal/mole for terminal methyl hydrogens and para-aromatic hydrogens, 0.0 Kcal/mole for axial sites on 6-membered aliphatic rings and ortho sites on aromatic rings, and −0.4 Kcal/mole for tertiary substituted aliphatic sites.

FIG. 7A illustrates a preferred process for generating the protrusion descriptor K_{R}. See 701. Protrusion is the extent to which the reactive atom lies inward or outward from the general surface of the molecule. First a vector vi is drawn from a reference point in the molecule to the reactive carbon. See 703.

\vec{V}ᵢ=\vec{X}ᵢ−\vec{X}_{ref}

The reference point it typically the center of mass of the molecule. The magnitude of the vector is then increased by the van der Waals radius of the carbon atom. See 705. Then the vector from the reference point to every other atom in the molecule is compared to this, beginning with operations 707 and 709, where the system sets a variable N equal to the number of atoms (707) and iterates over those sites (709). Iterative loop operation 709 initially sets an index value “i” equal to 1. It then determines whether the current value of i is greater than the value of N. If not, it performs various operations to compare the vectors for that atom.

The process draws the vector from the reference point to the atom. See 711. The component of that vector along the vector of the reactive carbon is then determined. See 713. The van der Waals radius of the atom being considered is then added to the magnitude of the vector along the reactive carbon. See 715. FIG. 7B illustrates a molecule with reference point 750, reactive carbon 751, vector to the reactive carbon 753, atom 755, and vector to the atom 757.

After all the atoms have been analyzed in this manner, an overall value is calculated to reflect the degree to which the atoms in the rest of the molecule render the reactive site inaccessible. See 717. In a preferred embodiment, this value is simply the Max value of the vector components along the reactive carbon vector. In essence, this means that the vector with the largest component along the reactive carbon vector is taken to be indicative of inaccessibility of the reactive site. If this Max value is greater than the magnitude of the reactive carbon vector, then a negative protrusion value results. If the Max value is less than the magnitude of the reactive carbon vector, then a positive protrusion value results.

This is the global protrusion value, R_{G} and is expressed by the following where vᵢ is the vector from reactive carbon i to the reference atom, Vⱼ is the vector from atom j to the reference point, rᵢ is the van der Waals radius of atom i, and rⱼ is the van der Waals radius of atom j.

$${R}_{G} = ( \vert {\vec{v}}_{i} \vert + {r}_{i} ) - {max}_{j \neq i} ( \frac{{\vec{v}}_{i} \cdot {\vec{v}}_{j}}{\vert {\vec{v}}_{i} \vert} + {r}_{j} )$$

The global protrusion value can be used to calculated the protrusion correction K_{R}. In a preferred embodiment, the process also derives a local and semi-local protrusion value. See 719. The local value is derived in the same manner at the global value, except that the atoms used are only those atoms within the chosen distance and that are conformationally rigid with respect to the origin. The semi-local value is also similarly derived, except that the atoms used are those within a chosen distance and that lie one rotatable bond away from the origin. The three protrusion values are then reversed in sign to reflect positive E_{A} increases for negative protrusion.

The process now has all the values needed to derive the protrusion curvature correction factor, K_{R}, where K_{R}=Y_{G} R_{G}+Y_{S} R_{S}+Y_{L} R_{L}. The overall global, semi-local and local protrusion curvature values are R_{G}, R_{S} and R_{L} respectively. The relative contributions of these values can be modified by the constants Y_{G} Y_{S}, and Y_{L}. In a preferred embodiment, Y_{G} is 1.0 and the other modifying constants are zero.

FIG. 8 illustrates a preferred process for generating the orientation correction factor K_{A}, in a preferred embodiment, an amphoteric correction factor. See 801. Because amphoteric interactions are specific both to the substrate and the enzyme, it is necessary to parameterize the process for a specific enzyme. In this embodiment, the process is parameterized for the CYP enzymes and in particular CYP3A4, but the process is adaptable to other enzymes. The active site of CYP3A4 can generally be characterized as having a highly polar environment right at its active site, but a hydrophobic environment in regions near the active site. Therefore, if a substrate molecule has a strong amphoteric moment, such that one side is generally polar and the other hydrophobic, it will tend to sit in one orientation in the active site of CYP3A4 polar-to-polar and hydrophobic-to-hydrophobic). If the reactive site in question sits on the hydrophobic end of such a molecule, then its reactivity will be diminished. If the reactive site is on the polar end, then its reactivity will be increased. Determining the amphoteric correction factor therefore involves two general steps, determining the amphoteric moment of the molecule, and then determining the component of that moment along the vector axis of the reactive site.

First the amphoteric moment of the molecule must be calculated beginning with to operations 803 and 805, where the system sets a variable N equal to the number of atoms (803) and iterates over those sites (805). Iterative loop operation 803 initially sets an index value “i” equal to 1. It then determines whether the current value of i is greater than the value of N. If not, it performs various operations to generate the amphoteric moment. For each atom in the molecule, the process draws the vector from a reference point, typically the center of mass, to the atom. See 807. That vector is multiplied by a function derived from the partial charge ƒ(qᵢ) and by the surface area of the atom si to yield the amphoteric moment. The simplest expression for ƒ(qᵢ) is just the absolute value, |qᵢ|. See 809 and 811. The probe radius used to determine the accessible surface area is typically that of the solvent molecule, such as water. Alternatively the surface area may be determined only from the van der Waals radius of the atom. The process of operations 803 through 811 can also be summarized by the following formula, where m is the amphoteric moment and v; is the vector to the atom:

\vec{v}ᵢ=\vec{x}ᵢ−\vec{x}_{ref}

$$m = {\sum }_{i = 1}^{N atoms} {S}_{l} f ( {q}_{i} ) {\vec{v}}_{i}$$

Typical magnitudes obtained are 0 to 100 Å e charge for non-amphoteric molecules and up to 450 Å e charge for strongly amphoteric molecules, using Gasteiger-Marsili partial equivalents of orbital energy. Note that the units for the units for these figures are Ångstroms×electron charge, where one electron charge is about 6×10⁻¹⁹ Coloumbs.

The vector of the reactive site is then drawn from the reference point to the reactive carbon. See 813. Taking the dot product of this vector with the amphoteric moment results in an amphoteric value for the reactive site. See 815. This can be modified with constants and parameters to yield the K_{A} amphoteric correction factor. See 817.

$${K}_{Ai} = \frac{{\vec{v}}_{i} \cdot m}{\vert {\vec{v}}_{i} \vert}$$

For instance has been found that large molecules tend to have an exaggerated amphoteric moment, and this influence can be included in the final K_{A} calculation.

The discussion will now turn to a more complex model for accessibility correction. In this model, as mentioned, the expression for corrected activation energy at each site may take the following form:

$${E}_{{A}_{corr}} = {E}_{A , 0} + {\sum }_{l}^{N steric descriptors} {C}_{l} {K}_{l} + {\sum }_{j}^{M orientation descriptors} {C}_{j} {K}_{j} .$$

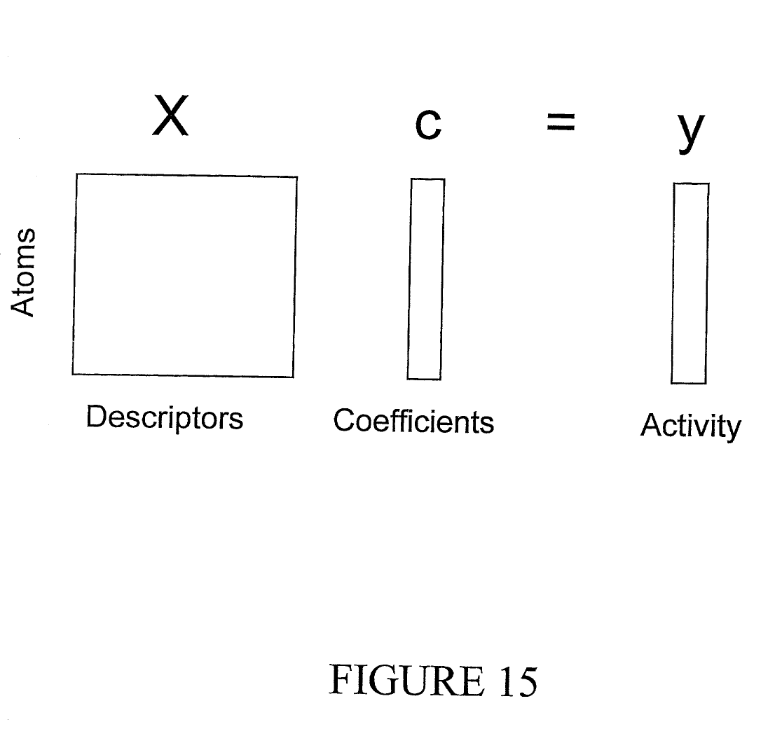

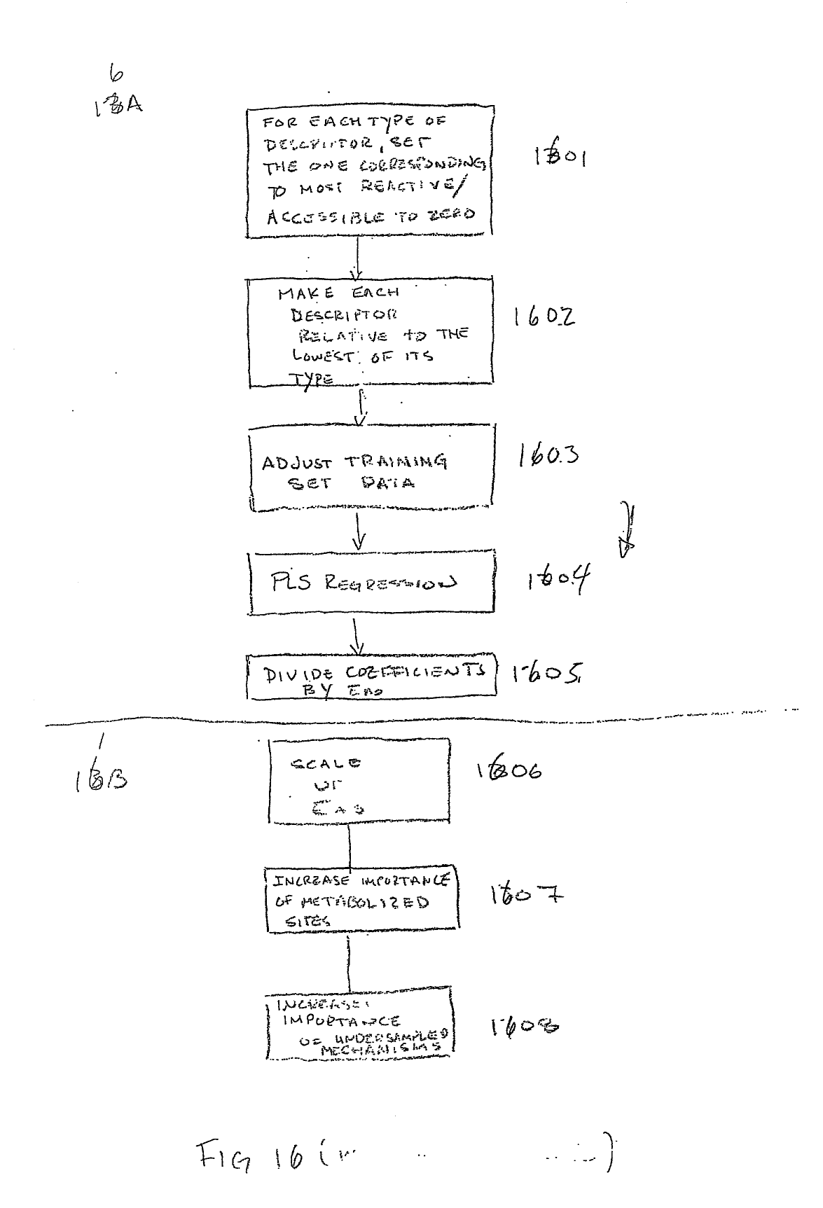

The Cᵢs and Cⱼs are coefficients for the steric and orientation descriptors, the Kᵢs and Kⱼs. Calculation of the coefficients C₁ is described in detail below with reference to FIGS. 15, 16A, and 16B.

FIG. 9 illustrates one general process 900 for generating the orientation accessibility descriptors, Kⱼs. First, the partial charge q₁ and the partial surface area Sᵢ of each atom i of the substrate molecule are generated in operation 901. The hydrophilicity or polarity pᵢ of each atom, a function of the partial charge and the partial surface area, is then generated in operation 903. Once the polarity is computed, the orientation accessibility descriptors are generated in operation 905. Depending on the isozyme that is being modeled, these descriptors may include distance-to-polar-regions, protrusion-weighted-distance-to-polar-regions, amphoteric moment, hydrophobicity, distance-to-charged-atoms, and other descriptors that help describe the effect of substrate orientation on metabolism by the isozyme. The orientation accessibility descriptors are generated for each potential reactive site. Operation 905 is then repeated for each conformation of the substrate. See 907. From the various conformations, a descriptor set is selected to represent the entire substrate.

The selection of the descriptor set can be done by many well-known methods. The descriptors could be averaged by a statistical method, such as Boltzmann weighting. In a preferred embodiment, the maximum value of each atomic descriptor is selected for the descriptor set. This method assumes that a molecule will assume conformations that correspond to more accessible and reactive sites and is less computationally intensive.

The partial charge of each atom can be calculated by many known quantum mechanical or empirical methods. For example, quantum mechanical techniques that estimate the partial charge of an atom from its electron density, such as Electrostatic Potential Fitting or Mulliken charges may be used. Alternatively, a method that generates the partial charge of an atom based on such empirical data as the electronegativity and ionization potential of the atom may be used. Examples of such methods include the Gasteiger method, the Gasteiger-Marsili method, the Huckell method, and the Gasteiger-Huckell method. In a preferred embodiment, a software routine generates the partial charges using the Gasteiger-Marsili method (Gasteiger, J., Marsili, M., Iterative Partial Equalization Of Orbital Electronegativity—A rapid Access To Atomic Charges, Tetrahedron Vol 36 p3219 1980). In a preferred embodiment the Gasteiger-Marsili method implemented in the MOE software package is used. MOE software is available from Chemical Computing Group, 1010 Sherbrooke St. West, Suite 910, Montreal, Quebec, Canada, H3A 2R7. Partial charges are expressed in units of electron charge, and typically range from −1 to 1.

Polarity is also a function of the partial surface area of the atom. Either the van der Waals surface area, which is a function only of the van der Waals radius of the atom and neighbor atoms and covalent bond lengths, or the solvent accessible surface area, a function of the van der Waals radius of atom, the radius of a probe atom, and three-dimensional conformation of the molecule can be used. The solvent accessible surface area is determined as described above. In a preferred embodiment the van der Waals surface area is used, and is generated using MOE software. The partial surface area can be expressed either as an absolute number or as a fraction of the total surface area of the molecule. The partial surface area of an atom may be stored from a previous calculation of a steric accessibility descriptor, in which case it could be retrieved from memory rather than recalculated.

After generating the partial charge qᵢ and the partial surface area Sᵢ of each atom i, the polarity pᵢ is generated in operation 902. For this purpose, a United Atom Model is preferably used for non-polar hydrogens, i.e. they are lumped onto the connecting atom before the polarity is calculated. In other embodiments, all atoms, including non-polar hydrogens, are considered separately. As mentioned above, unlike the steric accessibility descriptors, the orientation accessibility descriptors are isozyme specific. One way in which this is manifested is in the calculation of the polarity. For example, because the 2C9 enzyme prefers negatively charged substrates, only negatively charged atoms are considered polar in the model of metabolism by the 2C9 enzyme. Thus the polarity pᵢ of an atom i on the substrate is given by the following expression for metabolism of 2C9:

Pᵢ=qᵢSᵢδᵢ

where

δ=1 if qᵢSᵢ<0

0 if qᵢSᵢ≧0

Similarly for metabolism by the 2D6 enzyme, only the positive charges are included in calculation of the polarity:

pᵢ=qᵢSᵢδᵢ

where

δᵢ=1 if qᵢSᵢ<0

0 if qᵢSᵢ≦0

For metabolism by the 3A4 enzyme, both positively and negatively charged atoms are considered polar:

pᵢ=|qᵢ|Sᵢ

When positive and negative charges are considered, as with the 3A4 enzyme, an alternative method of calculating the polarity of each substrate atom is to decompose the partition coefficient, log P, of the molecule to find the atomic contributions to log P. One such method is described in Wildman, S. A., Crippen, G. M., Prediction of physicochemical parameters by atomic contributions, J. Chem. Inf. Comput. Sci., 39(5), 868-873 (1999) and is implemented in MOE software.

With the polarity of each potential reaction site in hand, the various orientation type descriptors can be calculated. Many descriptors represent a weighted average of some parameter centered a certain distance from the reaction site under consideration. FIG. 10 shows a cross-section of a shell centered at a distance 1010 from the reactive site 1000 with polar sites 1020, 1030, and 1040. In one embodiment, there are at least four sets of descriptors used to describe the relation between the location of the reactive site and the location of polar sites on the substrate: two sets that consider solely at the distance the reactive site to polar regions (distance-to-polar-regions descriptors) and two sets that consider the steric accessibility of the polar regions as well (protrusion-weighted-distance-to-polar-regions descriptors). The first set of distance-to-polar-regions descriptors K_{dtp}, captures the absolute amount of polarity within shells located certain distances from the reactive site. For example, the value of the 2 Å descriptor is the amount of polarity contained in a shell that is centered on a distance 2 Å from the reactive site. The 2 Å descriptor K_{dtp,2A} can be expressed by the following formula, where pᵢ is the hydrophilicity or polarity of the atom i, and w_{2A} is a weighting factor, a function of the distance from atom i to the reactive site:

$${K}_{dtp , 2 A} = {\sum }_{i}^{N atoms} {p}_{i} {w}_{2 A , i}$$

In a preferred embodiment, the weighting factor w is a Gaussian function centered on the distance from the reactive site, whose value approaches zero at distances of about 1.5 Å from the center of the shell. Thus the 2 Å descriptor captures polarity between approximately 1 Å and 3 Å from the reactive site, with the polarity at the center of the shell weighted more than that at the ends of the shell. In a preferred embodiment this set is comprised of seven such descriptors, centered on 2 Å, 4 Å, 6 Å, 8 Å, 10 Å, 12 Å, 14 Å, respectively. Various kernal weighting functions besides Gaussian functions may be employed. Examples include square kernals, triangular kernals, and the like.

The second set of distance-to-polar-regions descriptors K_{ndtp} captures the normalized amount of polarity within shells located certain distances from the reactive site. For example, the 2 Å descriptor is given by the following formula:

$${K}_{ndtp , 2 A} = \frac{{\sum }_{i}^{N atoms} {p}_{i} {w}_{2 A , i}}{{\sum }_{i}^{N atoms} {p}_{i}}$$

In a preferred embodiment this set is comprised of seven such descriptors, centered on 2 Å, 4 Å, 6 Å, 8 Å, 10 Å, 12 Å, 14 Å, respectively.

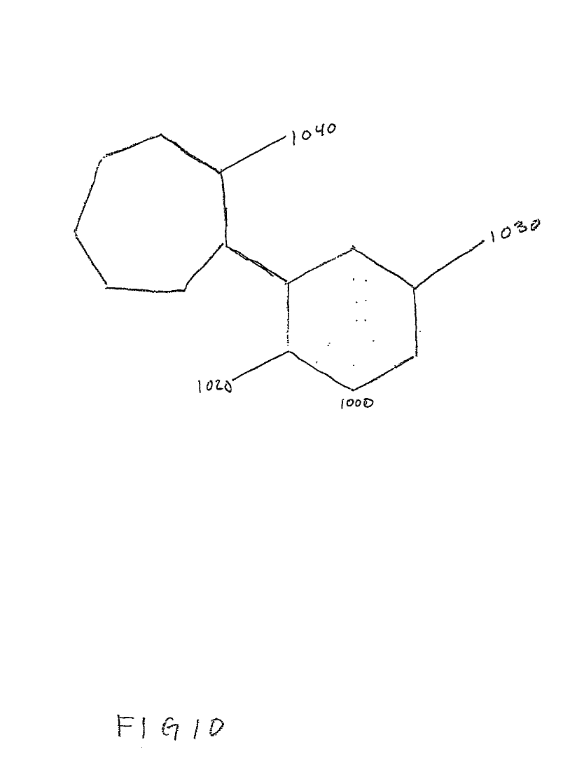

FIG. 11 shows a simplified schematic of a substrate molecule with reactive site 1100, polar site 1110 and polar site 1120. Although 1110 and 1120 are both polar, for some enzymes, polar site 1110 aids in metabolism more than polar site 1120 due to the protrusion of the 1110 site relative to the reactive site 1100. For the polar site to aid in metabolism, it, as well as the reactive site, must be accessible. The hydrophobic regions along the vector 1140 from site 1100 to site 1120 make the site 1120 less accessible (for certain enzymes) and so reduce the metabolizing effect of the hydrophilic region. In a preferred embodiment two protrusion-weighted-distance-to-polar-regions sets of descriptors account for this effect by considering the distance to the polar sites and the steric accessibility of those sites together. The first set K_{pwdtp} captures the absolute amount of polarity as weighted by the protrusion accessibility within shells located certain distances from the reactive site. Thus each descriptor is given by the following formula where Pᵢ is the polarity of the atom i, and w_{2A} is the weighting factor and P_{r i,r} is the protrusion of atom i relative to the reactive site r:

$${K}_{pwdtp , 2 A} = {\sum }_{i}^{N atoms} {p}_{i} {w}_{2 A , i} {p}_{{r}_{i , r}}$$

The protrusion p_{r i,r} is calculated as described above with atom i as the reference point. In a preferred embodiment the weight w is again a Gaussian function centered at the distance under consideration from the reactive site, although other weighting functions may be employed. In one embodiment, there are seven such descriptors in a preferred embodiment, centered on 2 Å, 4 Å, 6 Å, 8 Å, 10 Å, 12 Å, 14 Å, respectively.

A closely related set of descriptors describes the normalized protrusion-weighted distance to polar regions, at the same distances from the reactive site. For example the 2 Å descriptor is given by the following formula:

$${K}_{npwdtp , 2 A} = \frac{{\sum }_{i}^{N atoms} {p}_{i} {w}_{2 A , i} {p}_{{r}_{i , r}}}{{\sum }_{i}^{N atoms} {p}_{i} {\sum }_{i}^{N atoms} {p}_{{r}_{i , r}}}$$

Additional orientation accessibility descriptors can be used as well. One such descriptor is the amphoteric moment descriptor. This descriptor captures the where the reactive site of the substrate is in relation to its amphoteric moment. Calculation of the amphoteric moment descriptor is described above with reference to FIG. 8.

Other possible orientation accessibility descriptors include a hydrophobicity descriptor and a descriptor that measures proximity to charged atoms. The hydrophobicity descriptor K_{Hr} measures the hydrophobic character of the reactive carbon r by considering the partial charges and the partial surface areas of all the hydrogen atoms connected by a covalent bond to the reactive carbon. The descriptor K_{Hr} can be expressed by the following summation over all connected hydrogens, where Sⱼ is the partial surface area of hydrogen j, qⱼ is the partial charge of hydrogen j, and β is a parameter that determines when the charge is dominant in the equation:

$${K}_{Hr} = {\sum }_{j}^{N Hydrogens} {S}_{j} exp ( - β {q}_{j}^{2} )$$

β is set such that the Gaussian function exp(−βqⱼ²) goes to zero at a threshhold partial charge at which the atom is no longer considered hydrophobic. In a preferred embodiment this threshold is approximately ±0.3. Thus for 0.3<qⱼ<−0.3, the Gaussion function, and the contribution of the hydrogen j to the hydrophobicity, goes to zero. When the partial charge is low, the Gaussian function goes to one, and the contribution of hydrogen j to the hydrophobicity is proportional to the partial surface area of hydrogen j. In this specific embodiment the hydrophobicity enzyme is used for the 2C9 model, though it could also be used for other isozymes.

In a preferred embodiment, a proximity-to-charged-atoms descriptor, K_{Cr} is used in the metabolism by the 2C9 enzyme model. This descriptor looks at all the negatively charged atoms that are not nearest neighbors (i.e. coupled by a covalent bond) to the potential reactive site r. It is given by the following expression, where q, is the partial charge of atom i, qₜ is a threshold charge below which atoms are considered negatively charged, dᵣ₋ᵢ is the distance from potential reactive site r to atom i, and n is a nearest neighbor of reactive site r:

$${K}_{Cr} = {\sum }_{i \neq n}^{N atoms} \frac{{q}_{i}}{{d}_{r - i}} for {q}_{i} < {q}_{t}$$

In a preferred embodiment, qt is equal to −0.1 electron units. Additionally, rather than relying upon the connectivites to exclude nearest neighbors from the summation, a threshold distance dᵣ₋ᵢ can be specified. Typically specifying that dᵣ₋ᵢ be greater than approximately 2 Å will exclude nearest neighbors. Although used for metabolism by the 2C9 enzyme in the specific embodiment, the proximity-to-charged-atoms descriptor can be adapted for other enzymes.

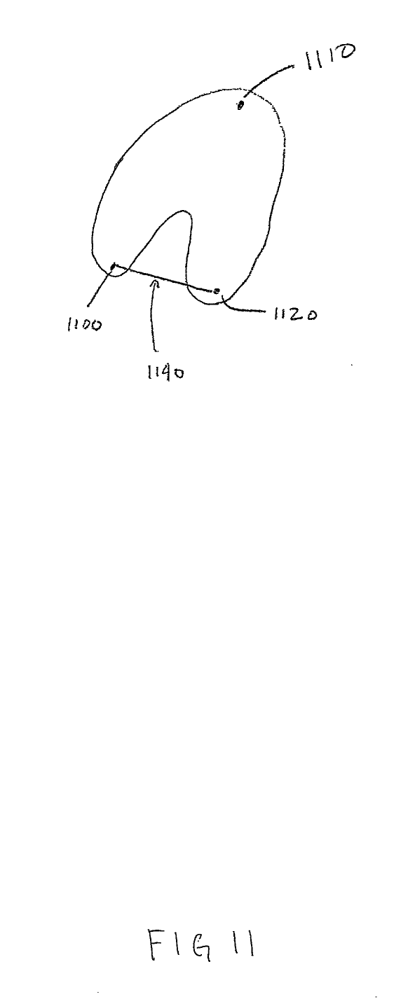

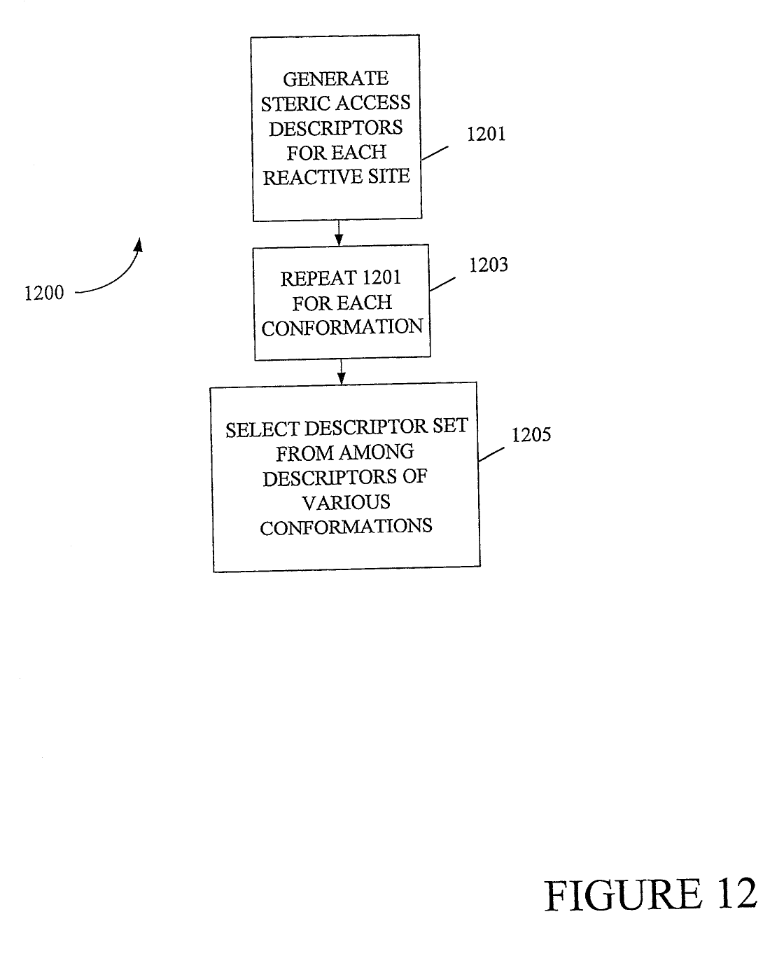

FIG. 12 illustrates one general process for generating the steric accessibility descriptors Kᵢs. First the steric access descriptors are generated for each reactive site in operation 1201. These descriptors may include surface area descriptors K_{SA}, parabolic curvature descriptors K_{P}, protrusion descriptors K_{R}, and extension descriptors K_{E}. The descriptors are generated for each conformation of the substrate in operation 1203. From the various conformations, a descriptor set is selected to represent the entire substrate in operation 1205.

The selection of the descriptor set can be done by many well-known methods. The descriptors could be averaged by a statistical method, such as Boltzmann weighting. In a preferred embodiment, the maximum value of each atomic descriptor is selected for the descriptor set. This method assumes that a molecule will assume conformations that correspond to more accessible and reactive sites and is less computationally intensive.

Generation of the surface area descriptors K_{SA} is explained in detail above with reference to FIG. 5. Generation of the parabolic curvature descriptors K_{P} is explained above with reference to FIGS. 6A and 6B. Generation of the protrusion descriptor K_{P} is explained above with reference to FIGS. 7A and 7B.

An important additional steric accessibility correction factor used in the complex model is extension accessibility. It is related to protrusion in that it compares the extension of an atom away from a reference point in the molecule relative to the extension of other atoms. The extension desriptor K_{E} is formulated as follows where vᵢ is the vector from reactive carbon i to the reference atom, vᵢ is the vector from atom j to the reference point, rᵢ is the van der Waals radius of atom i, and r, is the van der Waals radius of atom j.

$${K}_{E} = \frac{\vert {\vec{v}}_{i} \vert + {r}_{i}}{{max }_{j \neq l} ( \vert {\vec{v}}_{j} \vert + {r}_{j} )}$$

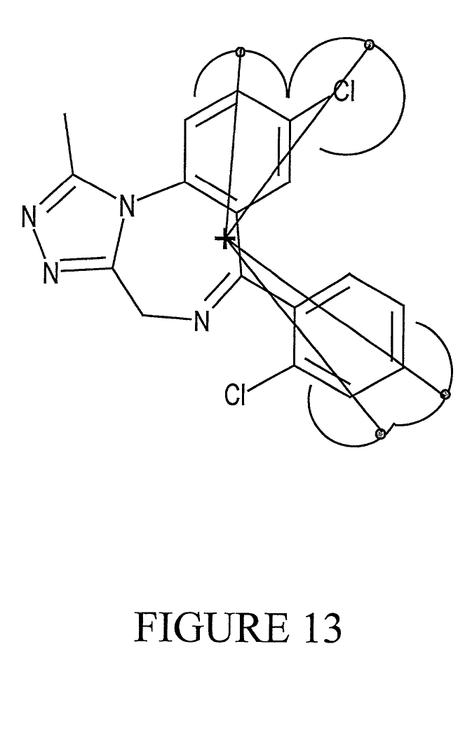

As the above equation indicates, in a preferred embodiment only the vectors and radii of the reactive carbon and the most extended atom are used in the final calculation of the extension descriptor. FIG. 13 illustrates a molecule with reference point 1350, reactive carbon 1351, vector to the reactive carbon 1353, atom 1355, and vector to the atom 1357.

### Generating A Correction Factors From Descriptors

This aspect of the invention may be viewed as a method of producing a model that accounts for accessibility parameters in predicting the lability of reactive sites on a chemical compound. The method may be characterized by the following sequence. First, the implementing system must obtain structural representations for a training set of chemical compounds. Second, for each of these chemical compounds, the system identifies one or more reactive sites pertinent to the model. Then, for each of these reactive sites, the system (i) determines whether metabolism at each site is experimentally observed; and (ii) characterizes the reaction site in terms of values for a plurality of chemical structural descriptors. These descriptors include at the electronic reactivity together with the steric and orientation descriptors described above. Finally, for all of the reaction sites, the system uses the site of metabolism information and the chemical structural descriptor values to obtain an expression for lability that sums contributions from each of the chemical structural descriptors.

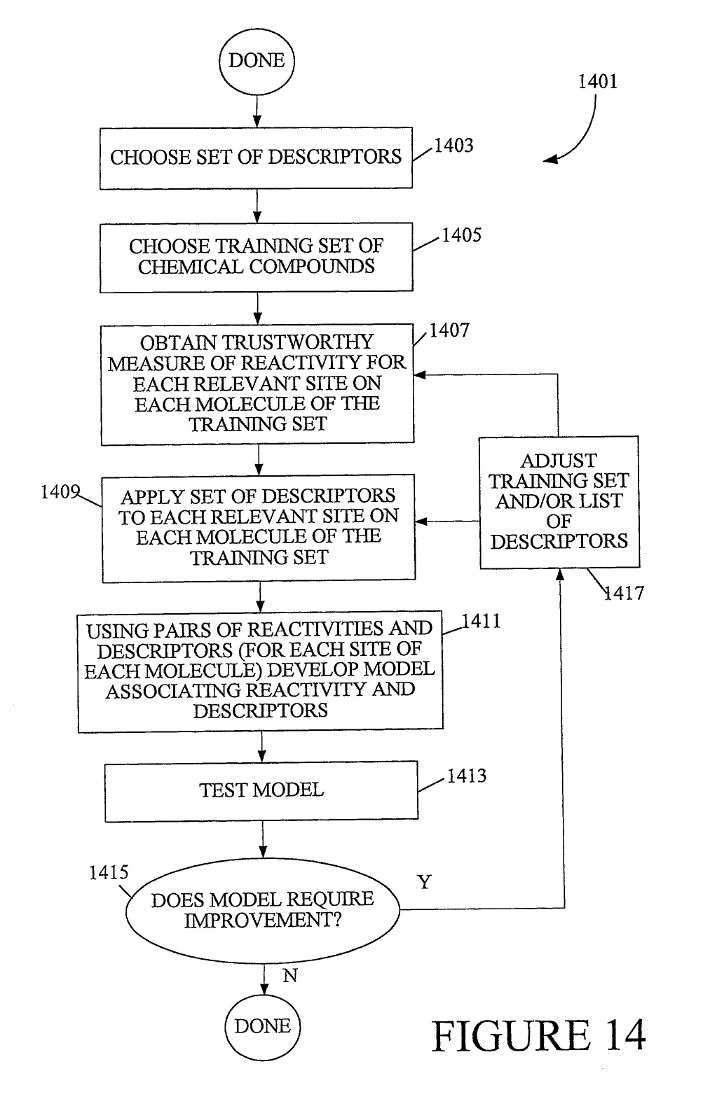

FIG. 14 presents a process flow diagram depicting typical operations that may be employed to generate a model in accordance with an embodiment of this invention. As depicted, a process 1401 begins with the choice of an appropriate set of structural descriptors for characterizing organic molecules. See 1403. Possibly, the set of descriptors is chosen for use in addressing a particular type or class of reactions (e.g., aromatic oxidation). This is because different classes of reaction may be impacted by accessibility factors very differently.

With the associated descriptors chosen, the next process operation involves obtaining information on an appropriate training set of organic molecules. See 1405. These molecules are chosen to provide a significant sampling of the types of structural characteristics and reactivities that the model is likely to encounter in practice. For each member of the training set, all potential reactive sites are identified. For each of these sites (on each molecule of the training set), the process obtains experimental information about whether each site is metabolized. See 1407.

The experimental site reactivity constitutes one component of each data point used to the construct the models of this invention. The other component is the descriptor values. Applying the set of descriptors identified at 1403, the process obtains actual values of those descriptors for each site on the training set compounds. See 1409. For example, one descriptor may be the weighted polarity 2 Å from the reactive site. The value of the descriptor is the actual numeric value of weighted polarity at that site. The procedure may obtain these descriptor values by analyzing the simple three-dimensional chemical structures of the members of the training set.

Once the descriptor values have been calculated, each relevant site of each member of the training set is now represented by a set of descriptor values and a trustworthy measure of reactivity. Then, using these data points, the process generates the actual model that associates reactivity with the descriptors. See 1411. The model may take the form of a simple expression including coefficients for each descriptor value. A detailed example of a model generating process will be described below.

With the model in hand, the process may test the model against a particular test set of molecules (or some actual field test molecules). See 1413. The molecules used in the test should have known sites of metabolism. The ability of the model to accurately predict these sites determines whether the model needs improvement. See 1415. Assuming that the model does a good job of predicting sites of metabolism, process 1401 is complete. Assuming that the model needs improvement, then a revised training set or list of descriptors is chosen. See 1417. From there, process control returns to 1407 or 1409 as appropriate. The revised set or list is chosen to handle the types of molecules or structural features that presented difficulty to the model.

In developing a model, one should carefully choose a training set. A large group of structurally diverse chemical compounds should be used. Generally, a training set member may be any compound that has been synthesized and has had its sites of metabolism characterized. The specific compounds chosen for the training set may also be focused on the chemical structural space relevant to the model. Thus, a useful training set may be comprised of compounds that possess an activity related to the activity of the compounds that will ultimately be screened with the model. For example, if the model pertains to drug metabolism, the training set compounds may be known drugs and/or drug-like compounds or other bioactive compounds.

The training set size depends in part on the amount of diversity among the members of the group. Structural “diversity” in the context of this invention means that the compounds of the set have a wide range of different functional groups and functional group environments. Such diversity may be obtained with a wide range of “scaffolds” and “building blocks” and/or a wide range of ring systems, substitutions, etc.

Since this invention pertains to models that predict reactivities of various sites on a compound, the training set should exhibit diversity in the structures of reactive sites represented. As indicated above, the “structure” of a site includes not only the particular atom or moiety at the site, but also the chemical and physical milieu of the site. Thus, for purposes of developing a diverse training set, a diverse set of site structures may include diversity in the neighboring atoms, ring systems, etc.

The training set may heavily emphasize groups of compounds and reactive site structures that exhibit widely ranging activation energies—to the extent that such compounds and structures exist. Because the reactivity of such sites may be significantly affected by slight and subtle structural changes, these sites can pose difficulties for the model. Therefore the training set may require numerous similar, but slightly varying, chemical structures.

In one approach to specifying a training set, a group of compounds is selected randomly or systematically based on building blocks, scaffolds, etc. After preliminarily analyzing a group of such compounds, their functional groups may be binned to identify a distribution of functional groups within the original training set. Those compounds that add little if anything to the pool of interesting functional groups may be discarded.

An expression for site reactivity (e.g., activation energy) may be obtained from any suitable data fitting technique. Generally, the expression is obtained by associating site reactivity with particular structural descriptors. Association represents an attempt to find a relationship between the two groups of variables. One set of variables is the dependent set of variables and these are a function of the other set, the independent set of variables. In this invention, the dependent variables are the extent to which each site undergoes an oxidation reaction and the independent variables are the structural descriptor values.

Examples of data fitting techniques that may be used with this invention include various regression techniques, partial least squares, principal component analysis, back-propagation neural networks and genetic algorithms. Principal component analysis is described in P. Geladi, Anal. Chim. Acta, 1986, 185, 1, which is hereby incorporated by reference.

A linear regression equation relates independent and dependent variables (Y=XB+e where Y is the dependent variable represented by a vector (i.e., reactivity of site of the training set members), X is the independent variable represented by a matrix (i.e., structural descriptors), B is the regression coefficient represented by a vector, and e is the residual). PLS (Projection to Latent Structures or Partial Least Squares) regression analysis is most commonly used with this invention because it can process large numbers of correlating descriptors while minimizing the risk of over-fitting.

In practice, one analyzes each member of the training set. For each member, one considers a list of potential reactive sites. Obviously, the sites of interest include only those that can undergo the reaction of the model at hand.

FIG. 15 shows the relationship between the atom descriptors and the activity of the atom. There are three types of descriptors: the electronic reactivity descriptor E_{A0}, the steric accessibility descriptors, and the orientation accessibility descriptors. To complete the model, it is necessary to determine the descriptor coefficients. This is done by using empirical data from training sets of atoms is substituted for the x (descriptors) and y (activity) variables. In a preferred embodiment, each activity variable yᵢ is assigned a value of either 1 or 0; 1 corresponding to a non-metabolized state, and 0 corresponding to a metabolized site. This assumes a uniform energy difference between the non-metabolized and metabolizes sites. If enough data is available, relative activity values could be used. The coefficients are then determined by a suitable technique. In a preferred embodiment, a partial least squares (PLS) regression is used, but any suitable regression or fitting method could be used.

The x-matrix in FIG. 15 is populated by all the descriptors from the descriptor sets of all the substrates of the training set. Each of the n rows represents an atom, where n is the total number of atoms in the training set. Each of the m columns represents a descriptor, where m is the total number of types of descriptors, i.e. the electronic reactivity descriptor E_{A0} plus the steric accessibility descriptors plus the orientation accessibility descriptors. Thus each xᵢⱼ represents descriptor j of atom i.

FIG. 16A shows the preferred process for determining the descriptor coefficients. Relative values of the descriptors are computed. For example, for the electronic reactivity descriptor, all values of E_{A0} are reduced by the lowest value of E_{A0}. Thus the lowest E_{A0} on the molecule, corresponding to the most electronically reactive site, is set to zero. This process is repeated for each descriptor in operations 1601 and 1602, with the values of each steric and orientation descriptor made relative to the value that corresponds to the greatest accessibility. The training set data is then adjusted in order to improve the PLS regression in operation 1603. The coefficients are then found from the PLS regression in operation 1604. Once the coefficients have been calculated by the PLS regression, all coefficients are divided by the coefficient E_{A0}, thereby rescaling them relative to the E_{A0} coefficient in operation 1605. This has the effect of converting all the terms into energy units.

FIG. 16B shows the adjustments to the training set data that may be necessary in order to generate good results from the PLS regression. First the values of the E_{A0} descriptor are scaled up by an arbitrary factor in operation 1606. This forces the PLS regression to accept E_{A0} as the first latent variable, and is necessary because the PLS regression is sensitive to the variance in the data; if there is a high degree of co-linearity amongst the data, the PLS method will capture it. This aspect of the PLS method has the effect of neglecting single descriptors such as E_{A0}. Scaling E_{A0} up ensures the PLS regression will sufficiently consider for the E_{A0} descriptor. The scale-up factor should typically be large enough that further increases do not affect the results of the regression. In a specific embodiment the scaling factor is typically on the order of 5 or 10.

The data corresponding to the metabolized sites is also adjusted in operation 1607. It is necessary to increase the importance of the undersampled data to ensure that it is not ignored. This is done by effectively increase the number of observations of the undersampled data. Since most of the sites on a molecule will not be metabolized, there will likely be far more there are far more observations of non-metabolized sites than of metabolized sites in the training set data. Thus the training set data must be adjusted to effectively increase the number of observations of metabolized sites. This can be accomplished either by software that weights the observations of by inputting the desired observations repeated times. The data is also or adjusted or weighted in order to give all the mechanisms of metabolism equal representation in operation 1608.

### Using Models to Approximate Site Reactivity

Generally, this aspect of the invention may be viewed as a method for predicting likelihood of metabolism of reactive sites on a chemical compound. Such method may be characterized as follows. First, the implementing system identifies a reactive site on the chemical compound. Second, it identifies values for a plurality of chemical structural descriptors for the reactive site. These descriptors are those described above. Third, the system calculates a metabolic likelihood value for the reactive site by summing terms of an expression, wherein the terms include or are derived from the chemical structural descriptors. The first three operations are repeated for more additional reactive sites of the chemical compound. Finally, the system outputs calculated metabolic likelihood values for the reactive sites on the chemical compound. The system may simultaneously display the calculated metabolic likelihood values for all reactive sites on the compound.

The models of this invention can also be used in conjunction with, or to supplement, the more rigorous quantum chemical models. The quantum mechanical models may provide the values of the electronic component of site reactivity, for example.

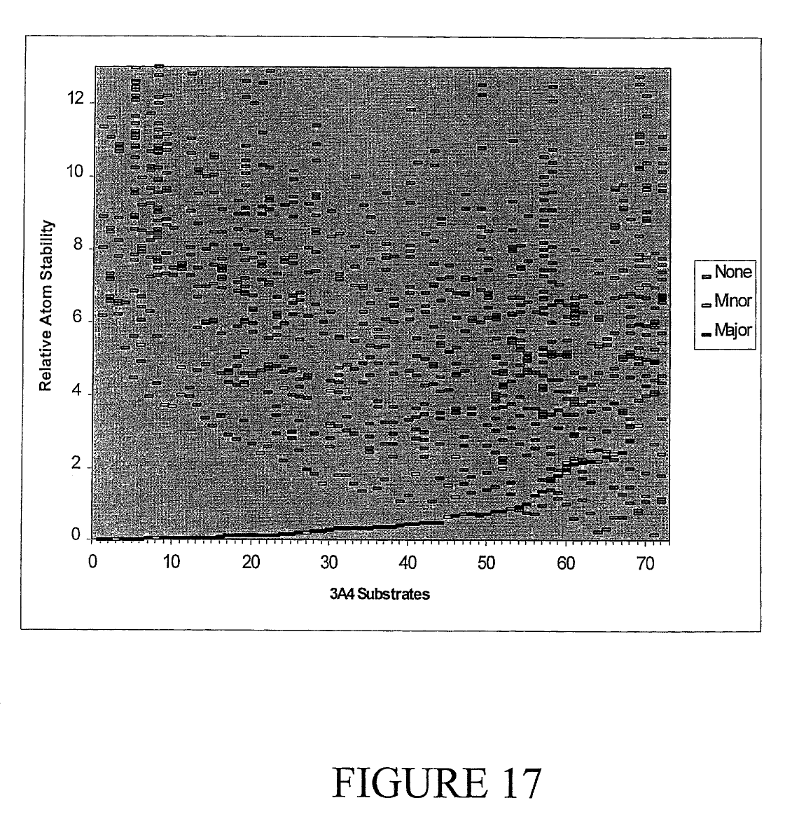

FIG. 17 is an example of a relative atom stability plot for substrates metabolized by the 3A4 enzyme. The relative atom stability can be then be compared to empirical results. This information can then be used to derive a confidence score for the results predicted by the model. The relative atom stability of atom i is given by the following expression:

$${k}_{1} = exp ( \frac{- {E}_{A , corr , i}}{RT} )$$

relative stability of atom

$$i = \frac{{k}_{i}}{{\sum }_{i}^{N atoms} {k}_{i}}$$

Thus the least stable and likeliest site to react on the substrate, as predicted by the model, will have the lowest relative atom stability. Each x-axis unit in FIG. 17 represents a substrate. Each point represents a potential reactive site on the substrate, the y-value corresponding to the relative atom stability. Empirical data can be represented on the plot by, for example, colors of the data points: a red point indicating a major site of metabolism, a yellow point a minor site of metabolism, and a gray point a non-metabolized site. The data in FIG. 17 is organized with the substrate with largest energy gap between the likeliest to react and the next likeliest to react sites, as predicted by the model, at the lowest x-value. Since the E_{A},_{corr} values returned by the model, may have a values between 1 (corresponding to a non-metabolized state) and 0 (corresponding to a metabolized state), a threshold value of E_{A,corr} at which a site is declared metabolized can be chosen. It can be seen from FIG. 17 that the number of sites incorrectly predicted to be metabolized (false positives) and the number of sites incorrectly predicted to be non-metabolized (false negatives) for a given threshold value is easily found from the relative site stability data. This information can then be used to derive a confidence score for the predicted results. It can be seen from FIG. 17 that as the threshold value increases, the number of false positives increases and the number of false negatives decreases.

### Hardware and Software

Generally, embodiments of the present invention employ various processes involving data stored in or transferred through one or more computer systems. Embodiments of the present invention also relate to an apparatus for performing these operations. The processes are those described above, such as generating accessibility descriptors and correction factors, generating electronic components of reactivity, predicting site specific reactivity of compounds, generating models that account for both electronic and accessibility components of site reactivity, etc. This apparatus may be specially constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media or computer program products that include program instructions and/or data (including data structures) for performing various computer-implemented operations in accordance with this invention. The program instructions may specify various operations and procedures described above, such as generating accessibility descriptors and correction factors, generating electronic components of reactivity, predicting site specific reactivity of compounds, generating models that account for both electronic and accessibility components of site reactivity, etc. Examples of computer-readable media include, but are not limited to, magnetic media such as hard disks, floppy disks, and magnetic tape; optical media such as CD-ROM devices and holographic devices; magneto-optical media; semiconductor memory devices, and hardware devices that are specially configured to store and perform program instructions, such as read-only memory devices (ROM) and random access memory (RAM), and sometimes application-specific integrated circuits (ASICs), programmable logic devices (PLDs) and signal transmission media for delivering computer-readable instructions, such as local area networks, wide area networks, and the Internet. The data and program instructions of this invention may also be embodied on a carrier wave or other transport medium. Examples of program instructions include both machine code, such as produced by a compiler, and files containing higher level code that may be executed by the computer using an interpreter.

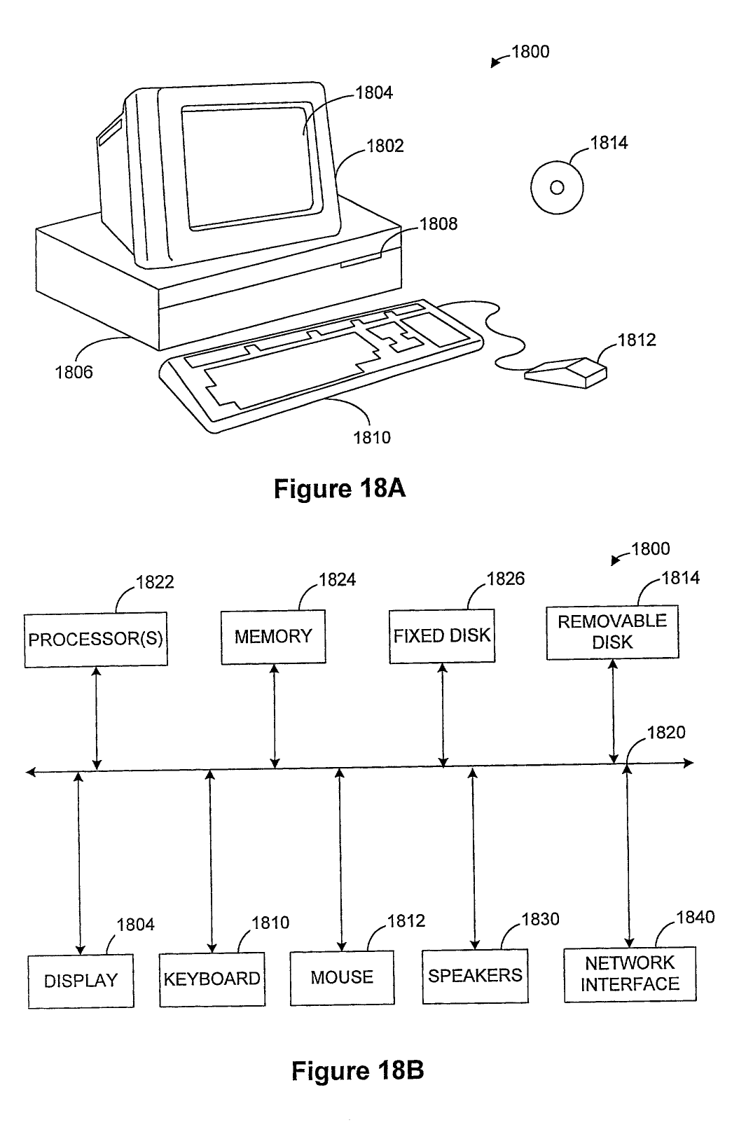

FIGS. 18A and 18B illustrate a computer system 1800 suitable for implementing embodiments of the present invention. FIG. 18A shows one possible physical form of the computer system. Of course, the computer system may have many physical forms ranging from an integrated circuit, a printed circuit board and a small handheld device up to a very large super computer. Computer system 1800 includes a monitor 1802, a display 1804, a housing 1806, a disk drive 1808, a keyboard 1810 and a mouse 1812. Disk 1814 is a computer-readable medium used to transfer data to and from computer system 1800.

FIG. 18B is an example of a block diagram for computer system 1800. Attached to system bus 1820 are a wide variety of subsystems. Processor(s) 1822 (also referred to as central processing units, or CPUs) are coupled to storage devices including memory 1824. Memory 1824 includes random access memory (RAM) and read-only memory (ROM). As is well known in the art, ROM acts to transfer data and instructions uni-directionally to the CPU and RAM is used typically to transfer data and instructions in a bi-directional manner. Both of these types of memories may include any suitable of the computer-readable media described below. A fixed disk 1826 is also coupled bi-directionally to CPU 1822; it provides additional data storage capacity and may also include any of the computer-readable media described below. Fixed disk 1826 may be used to store programs, data and the like and is typically a secondary storage medium (such as a hard disk) that is slower than primary storage. It will be appreciated that the information retained within fixed disk 1826, may, in appropriate cases, be incorporated in standard fashion as virtual memory in memory 1824. Removable disk 1814 may take the form of any of the computer-readable media described below.

CPU 1822 is also coupled to a variety of input/output devices such as display 1804, keyboard 1810, mouse 1812 and speakers 1830. In general, an input/output device may be any of: video displays, track balls, mice, keyboards, microphones, touch-sensitive displays, transducer card readers, magnetic or paper tape readers, tablets, styluses, voice or handwriting recognizers, biometrics readers, or other computers. CPU 1822 optionally may be coupled to another computer or telecommunications network using network interface 1840. With such a network interface, it is contemplated that the CPU might receive information from the network, or might output information to the network in the course of performing the above-described method steps. Furthermore, method embodiments of the present invention may execute solely upon CPU 1822 or may execute over a network such as the Internet in conjunction with a remote CPU that shares a portion of the processing.

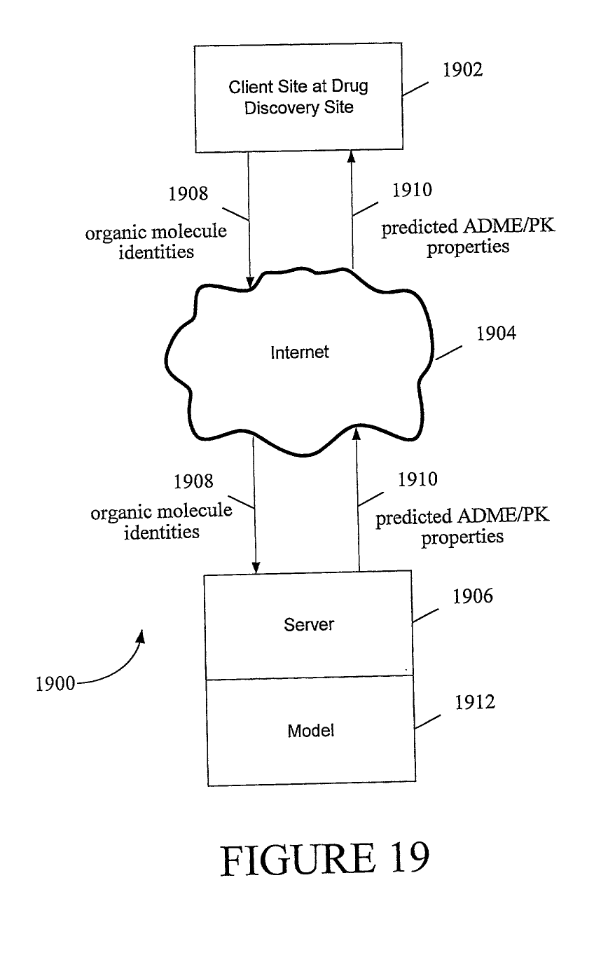

FIG. 19 is a schematic illustration of an Internet-based embodiment of the current invention. See 1900. According to a specific embodiment, a client 1902, at a drug discovery site, for example, sends data 1908 identifying organic molecules 1908 to a processing server, 1906 via the Internet 1904. The organic molecules are simply the molecules that the client wishes to have analyzed by the current invention. At the processing server 1906, the molecules of interest are analyzed by a model 1912, which predicts site-by-site reactivities in accordance with the current invention. After the analysis, the calculated ADMET/PK properties 1910, are sent via the Internet 1904 back to the client 1902. The computer system illustrated in FIGS. 8A and 8B is suitable both for the client 1902 and the processing server 1906. In a specific embodiment, standard transmission protocols such as TCP/IP (transmission control protocol/internet protocol) are used to communicate between the client 1902 and processing server 1906. Standard security measures such as SSL (secure socket layer), VPN (virtual private network) and encryption methods (e.g., public key encryption) can also be used.

Although various details have been omitted for brevity's sake, obvious design alternatives may be implemented. Therefore, the present examples are to be considered as illustrative and not restrictive, and the invention is not to be limited to the details given herein, but may be modified within the scope of the appended claims.

What is claimed is:

1. A method of predicting the susceptibility of a reactive site on a molecule to metabolism, the method comprising:
a) receiving a value of an electronic contribution to reactivity for the site;
b) calculating an accessibility correction factor for the site;
c) applying the accessibility correction factor to the initial activation energy value to generate a new reactivity value for the site; and
d) outputting the new reactivity value for the site.

2. The method of claim 1 wherein (a), (b), (c), and (d) are repeated for multiple reactive sites on the substrate molecule.

3. The method of claim 2, further comprising determining which of the multiple reactive sites is most likely to undergo metabolism.

4. The method of claim 1 wherein the molecule accessibility correction factor is calculated for a cytochrome P450 enzyme.

5. The method of claim 1, wherein the accessibility correction factor reflects how the molecule can orient itself in a metabolic enzyme.

6. The method of claim 1, wherein the accessibility correction factor reflects steric constraints on the accessibility of the site.

7. The method of claim 1, wherein applying the accessibility correction factor to the initial activation energy value comprises summing the accessibility correction factor and the initial activation energy.

8. The method of claim 1, wherein the accessibility correction factor is a function of one or more accessibility descriptors.

9. The method of claim 1, wherein the one or more accessibility descriptors are selected from the group consisting of orientation accessibility descriptors and combinations thereof.

10. The method of claim 1, wherein the one or more accessibility descriptors are selected from the group consisting of steric accessibility descriptors and combinations thereof.

11. The method of claim 1, wherein applying the accessibility correction factor to the initial activation energy value comprises employing the following expression:
$${E}_{Acorr} = {E}_{A , 0} + {\sum }_{i}^{N steric descriptors} {C}_{i} {K}_{i} + {\sum }_{j}^{M orientation descriptors} {C}_{j} {K}_{j} .$$
wherein E_{Acorr} is the new reactivity value for the site, wherein E_{A0} is the electronic contribution to reactivity for the site, wherein the Cᵢs and Cⱼs are coefficients for steric and orientation descriptors, respectively, and wherein the Kᵢs and Kⱼs are steric and orientation descriptors.

12. The method of claim 1, wherein the accessibility correction factor reflects an amphoteric effect manifest the molecule.

13. The method of claim 12, wherein the amphoteric effect is calculated by
a) calculating a surface area of each atom on the molecule;
b) calculating a partial charge of each atom on the molecule;
c) calculating an amphoteric moment;
d) extending a vector from a reference point to the reactive site on the molecule;
e) outputting an amphoteric correction factor.

14. The method of claim 1, wherein the accessibility correction factor corrects for a surface area accessibility at the reactive site on the molecule.

15. The method of claim 1, wherein calculating the surface area accessibility correction factor comprises:
a) choosing a probe radius;
b) determining the exposed surface area of an atom in the reactive site;
c) comparing the exposed surface area to a reference value; and
d) outputting a surface area correction factor.

16. The method of claim 15 wherein the probe radius is the radius of a solvent molecule.

17. The method of claim 15 wherein the reference value is the surface area of a hydrogen in a methyl group on an aliphatic chain.

18. The method of claim 15 wherein the reference value is the surface area of a carbon in an aromatic group.

19. The method of claim 1, wherein the accessibility correction factor reflects a parabolic curvature effect at the reactive site on the molecule.

20. The method of claim 19, wherein calculating the parabolic curvature accessibility correction factor comprises:
a) identifying a point on or near one of the atoms in the reactive site;
b) parameterizing at least one parabola using a point on or near an atom that is within about 10A of the atom in the reactive site; and
c) outputting a parabolic curvature correction factor.

21. The method of claim 1, wherein the accessibility correction factor reflects protrusion accessibility effects at the reactive site on the molecule.

22. The method of claim 21, wherein calculating the protrusion accessibility correction factor comprises:
a) choosing an atom in the reactive site;
b) extending a vector from a standard point in the molecule to the atom;
c) assigning a score to the vector; and
d) outputting an protrusion accessibility correction factor.

23. The method of claim 1, wherein the accessibility correction factor reflects extension accessibility effects at the reactive site on the molecule.

24. The method of claim 23, wherein calculating the extension accessibility correction factor comprises:
a) choosing an atom in the reactive site;
b) extending a vector from a standard point in the molecule to the atom;
c) assigning a score to the vector; and
d) outputting an extension accessibility correction factor.

25. The method of claim 1, wherein the accessibility correction factor reflects distance to polar regions effects at the reactive site of the molecule.

26. The method of claim 25, wherein calculating the distance to polar regions effects accessibility correction factor comprises:
a) calculating the polarity of each atom on the molecule;
b) identifying at least one range of distances from the reactive site;
c) determining the amount of polarity within each range;
d) outputting a distance to polar regions correction factor for each range.

27. The method of claim 25, wherein the distance to polar regions effects correction factor is weighted by the protrusion of atoms in the range.

26. The method of claim 1, wherein the accessibility correction factor reflects hydrophobicity effects at the reactive site of the molecule.

27. The method of claim 26, wherein calculating the hydrophobicity effects accessibility correction factor comprises:
a) identifying a reactive atom in the reactive site;
b) identifying atoms connected to the reactive atom;
c) calculating the surface area of at least some of the connected atoms;
d) calculating the partial charge of at least some of the connected atoms;
e) outputting a hydrophobicity effects correction factor.

28. The method of claim 1, wherein the accessibility correction factor reflects the distance to charged atoms effects at the reactive site of the molecule.

29. The method of claim 28, wherein calculating the distance to charged atom effects accessibility correction factor comprises:
a) identifying a reactive atom in the reactive site;
b) calculating the partial charge of atoms on the molecule;
c) identifying a threshold charge;
d) calculating the distance from the reactive atom on the molecule;
e) identifying a threshold distance or threshold degree of connectivity to the reactive atom;
f) outputting a distance to charged atoms effects accessibility correction factor.
